SEQUENCE LISTING (8 sequences)

SEQ ID NO: 1 - protein, 1301 aa, Homo sapiens
MTMGDMKTPDFDDLLAAFDIPDMVDPKAAIESGHDDHESHMKQNAHGEDDSHAPSSSDVGVSVIVKNVRNIDSSEGGEKDGHNPTGNGLHNGFLTASSLDSYSKDGAKSLKGDVPASEVTLKDSTFSQFSPISSAEEFDDDEKIEVDDPPDKEDMRSSFRSNVLTGSAPQQDYDKLKALGGENSSKTGLSTSGNVEKNKAVKRETEASSINLSVYEPFKVRKAEDKLKESSDKVLENRVLDGKLSSEKNDTSLPSVAPSKTKSSSKLSSCIAAIAALSAKKAASDSCKEPVANSRESSPLPKEVNDSPRAADKSPESQNLIDGTKKPSLKQPDSPRSISSENSSKGSPSSPAGSTPAIPKVRIKTIKTSSGEIKRTVTRVLPEVDLDSGKKPSEQTASVMASVTSLLSSPASAAVLSSPPRAPLQSAVVTNAVSPAELTPKQVTIKPVATAFLPVSAVKTAGSQVINLKLANNTTVKATVISAASVQSASSAIIKAANAIQQQTVVVPASSLANAKLVPKTVHLANLNLLPQGAQATSELRQVLTKPQQQIKQAIINAAASQPPKKVSRVQVVSSLQSSVVEAFNKVLSSVNPVPVYIPNLSPPANAGITLPTRGYKCLECGDSFALEKSLTQHYDRRSVRIEVTCNHCTKNLVFYNKCSLLSHARGHKEKGVVMQCSHLILKPVPADQMIVSPSSNTSTSTSTLQSPVGAGTHTVTKIQSGITGTVISAPSSTPITPAMPLDEDPSKLCRHSLKCLECNEVFQDETSLATHFQQAADTSGQKTCTICQMLLPNQCSYASHQRIHQHKSPYTCPECGAICRSVHFQTHVTKNCLHYTRRVGFRCVHCNVVYSDVAALKSHIQGSHCEVFYKCPICPMAFKSAPSTHSHAYTQHPGIKIGEPKIIYKCSMCDTVFTLQTLLYRHFDQHIENQKVSVFKCPDCSLLYAQKQLMMDHIKSMHGTLKSIEGPPNLGINLPLSIKPATQNSANQNKEDTKSMNGKEKLEKKSPSPVKKSMETKKVASPGWTCWECDCLFMQRDVYISHVRKEHGKQMKKHPCRQCDKSFSSSHSLCRHNRIKHKGIRKVYACSHCPDSRRTFTKRLMLEKHVQLMHGIKDPDLKEMTDATNEEETEIKEDTKVPSPKRKLEEPVLEFRPPRGAITQPLKKLKINVFKVHKCAVCGFTTENLLQFHEHIPQHKSDGSSYQCRECGLCYTSHVSLSRHLFIVHKLKEPQPVSKQNGAGEDNQQENKPSHEDESPDGAVSDRKCKVCAKTFETEAALNTHMRTHGMAFIKSKRMSSAEK

SEQ ID NO: 2 - DNA, 6493 bp, Homo sapiens
gccatgtttcaatctggccccagtggctttttctctgaaagcaaacgtgtgtcttttacaccagggctttctccccaccccagggggtgtcttccatccttttgtggctcagttgaaggcgaaaagggctccaaaccactaactaaccagaggagagccccttcttccacctccagggagaatttcagatttaatttgtccgaagatagcgtgctctcttcttactcatttgccatcattacgaggaaaacaaaccaccaccttggcttcaagatcctgggtagaggctcacggtcttttcaaccatctttggcgaggccttgcttccttccactcgaggtatgttctgtcttgtgctttttcttttagaagctactaaagggtgttggggatgcttctgactattatgaaggccaaaaggcctgttgactggggctgcttttaaccctttcctatttgctgagaatgcagccgtgtgacagtaactg[450 bp omitted]cgatctccagtgctgaagagtttgatgacgacgagaagattgaggtggatgacccccctgacaaggaggacatgcgatcaagcttcaggtcgaatgtgttgacggggtcggctccccagcaggactacgataagctgaaggcactcggaggggaaaactccagcaaaactggactctctacgtcaggcaatgtggagaaaaacaaagctgttaagagagaaacagaagccagttctataaacctgagtgtttatgaaccttttaaagtcagaaaagcagaggataaattgaaggaaagctctgacaaggtgctggaaaacagagtcctagatgggaagctgagctccgagaagaatgacaccagcctccccagcgttgcgccatcaaagacaaagtcgtcctccaagctctcgtcctgcatcgctgccatcgcggctctcagcgctaaaaaggcggcttcagactcctgcaaagaaccagtggccaattcgagggaatcctccccgttaccaaaagaagtaaatgacagtccgagagccgctgacaagtctcctgaatcccagaatctcatcgacgggaccaaaaaaccatccctgaagcaaccggatagtcccagaagcatctcaagtgagaacagcagcaaaggatccccgtcctctcccgcagggtccacaccagcaatccccaaagtccgcataaaaaccattaagacatcttctggggaaatcaagagaacagtgaccagggtattgccagaagtggatcttgactctggaaagaaaccttccgagcagacagcgtccgtgatggcctctgtgacatcccttctgtcgtctccagcatcagccgccgtcctttcctctccccccagggcgcctctccagtctgcggtcgtgaccaatgcagtttcccctgcagagctcacccccaaacaggtcacaatcaagcctgtggctactgctttcctcccagtgtctgctgtgaagacggcaggatcccaagtcattaatttgaagctcgctaacaacaccacggtgaaagccacggtcatatctgctgcctctgtccagagtgccagcagcgccatcattaaagctgccaacgccatccagcagcaaactgtcgtggtgccggcatccagcctggccaatgccaaactcgtgccaaagactgtgcaccttgccaaccttaaccttttgcctcagggtgcccaggccacctctgaactccgccaagtgctaaccaaacctcagcaacaaataaagcaggcaataatcaatgcagcagcctcgcaaccccccaaaaaggtgtctcgagtccaggtggtgtcgtccttgcagagttctgtggtggaagctttcaacaaggtgctgagcagtgtcaatccagtccctgtttacatcccaaacctcagtcctcccgccaatgcagggatcacgttaccgacgcgtgggtacaagtgcttggagtgtggggactcctttgcacttgaaaagagtctgacccagcactacgacagacggagcgtgcgcatcgaagtaacgtgcaaccattgtacaaagaacctcgttttttacaacaaatgcagcctcctttcccatgcccgtgggcataaggagaaaggggtggtaatgcaatgctcccacttaattttaaagccagtcccagcagatcaaatgatagtttctccgtcaagcaatacttccacttcaacttccactcttcagagccctgtgggagctggcacacacactgtcacaaaaattcagtctggcataactgggacagtcatatcggctccttcaagcactcccatcaccccagccatgcccctagatgaagacccctccaaactgtgtagacatagtctaaaatgtttggagtgtaatgaagtcttccaggacgagacatcactggctacacatttccagcaggctgcagatacgagtggacaaaagacttgcactatctgccagatgctgcttcctaaccagtgcagttatgcatcacaccagagaatccatcagcacaaatctccctacacctgccctgagtgtggggccatctgcaggtcggtgcacttccagacccacgtcaccaagaactgtctgcactacacgaggagagttggttttcgatgtgtgcattgcaatgttgtgtactctgatgtggctgctctgaagtctcacattcaaggttctcactgtgaagtcttctacaagtgtcctatttgtccaatggcgtttaagtctgccccaagcacacattcccacgcctacacacagcatcctggcatcaagataggagaaccaaaaataatatataagtgttccatgtgcgacactgtgttcaccctgcaaaccttgctgtatcgccactttgaccaacacattgaaaaccagaaggtgtctgttttcaagtgtccagactgttctcttttatatgcacagaagcaacttatgatggaccatatcaagtctatgcatggaacattgaaaagtattgaagggcctccaaacttgggtataaacttgcctttgagcattaagcctgcaactcaaaattcagcaaatcagaacaaagaggacaccaaatccatgaatgggaaagagaaattggaaaagaaatctccatctcctgtgaaaaaatcaatggaaaccaagaaagtggccagtcctgggtggacgtgttgggagtgtgactgcctgttcatgcagagagatgtgtacatatcccacgtgaggaaggagcacgggaagcaaatgaagaaacacccctgccgccagtgtgacaagtctttcagctcgtcccacagcctgtgccggcacaaccggatcaagcacaaaggcatcaggaaagtgtacgcctgctcgcactgcccagactccagacgtacctttaccaaacgtttgatgctggagaagcacgtccagctgatgcatggcatcaaggaccctgacctgaaagaaatgacagatgccaccaatgaggaggaaacagaaataaaagaagacactaaggtccccagtcccaagcggaagttggaagaaccagttctggagttcaggcctccccgaggagcaatcactcaaccactgaaaaagctgaaaatcaatgtttttaaggttcacaagtgtgccgtgtgtggcttcaccaccgaaaacctgctgcaattccacgaacacatccctcagcacaaatcggatggttcttcctaccagtgccgggagtgtggcctctgctacacgtctcacgtctctctgtccaggcacctcttcatcgtacacaagttaaaggaacctcagccagtgtccaagcaaaatggggctggggaagataaccaacaggagaacaaacccagccacgaggatgaatcccctgatggcgccgtgtcagacagaaagtgcaaagtgtgcgcaaaaacttttgaaactgaagctgccttaaatactcacatgcggacacacggcatggccttcatcaaatccaaaaggatgagctcagccgagaaatagccacagatgctccatgaggaaaatccctgtccacattggaataaaaaagacatttttgttacaaagtttgcagtataatagagttaacagtactgtctaggctgttgcaatatattctctttcaatgtaccttccttcacctcgtcgtatatatcctcgataagtattaaaacagtatttgagtttaaaagagtttgtatatatttaaatgaataactttttatactctttgttacatgtttgtatcagtatttagtggaaaaccatttgagttgttttgggttagaatttttctttttgtactgtttctttaaaacagagttcttagtaacaggggcagttcctgaattcaaataaaccattttgtatgtttggattttgaatgggttaactaattacaggctaaaataatgccttttttagtgtttttaatttttagaattcactacataaattgtaagtaattgtgggtctcaaaaacactaggaacttttaagtgtcttagcacttcctcgatgtgcctgccctgagggagtgagttcacatttgagacaactgcactccagtgtggacgtgcctttgtcttcaggccatgccgaagggtgtttaaagcagtcttgcaggtcgctcctttcccagccgtggataaaaactgaagctaggaatctaataaggaatgctgatttcctcagttccattttgaggaatggggaaggctattctaaagaaaaaaatgggatttgttttctcggcagatctgcaaggctggctttaagagcacaaggagggaaagtaacgaaagggctggactactataaaagttacaaatacgtagttagaccaatagatttatatagtcaggtttttgtcatgtaatttattaactaactattacagaaacacagctaagaatatcaagtatttctctggctcttgacagaaaaaaatcagttgacttaaccctttgctgtcaaaagagttggcgtttcctgttctgggtgctactgccaaacgttatggtacttagagtcgggatgcacaacttcaaccaccgacttatcaatgcagccgcctgtgtattgcaattggccgttaccttaagcactgagccacccgggtttagttcagccatttcaagaagtatatttaacgtcggtagttctgctttattaaaatgcagcagaggtactcttctgtcccttccgtttatagttctctgagagagttctattttttggttttgttttgtgttttcttttgcattttgtatcttgtatttatccctgaacatgttttgtaccttttttttttttttttttaagaaaaggaattcttttgtgtatatatagatacttgcatgatatactgtagtcaatgttcggttcctcaaaaggtcttgctgctgtcaggtgttatgcactccatccatcataactgtatgaaacacatttcatatgtaaataaacgtgggacatttggcccttgtgcttctgtgagagaattattgatggtgggtctctgacatctttgtgaagtttgggaagtaattaattgcagcgacaagctacagggtgttgcagaattcttcccactcagaagaatggcatattcgttctcattagtaatcagctattttgtcactttcttgttgactccatcagtacatgggtacaatccgagggtgtgaatttcagcttgaaattccattgctgttccttgttttgtttgtattgctctaagttgtattcataatagcactttcatatgtttctgcatttgaaccttgcaataagcctgtgtggtaggccacataggtccgaataacctagttttacagttgagggagctgagctcagattcagttctttgccgaagccctcatagctggtaagtggctttgcatattagaacccaaatattttgctctctaaatctaatgctcgctctatgtggttatgtacatattgacaaatattcatttattcaacaaataaaaagtatgtacaaaacaaaaaaaaaaaaaaaaaaaaaaaaaaaaaa

SEQ ID NO: 3 - DNA, 6036 bp, Artificial Sequence
  Description of Artificial Sequence Synthetic polynucleotide
[391 bp omitted]cgatctccagtgctgaagagtttgatgacgacgagaagattgaggtggatgacccccctgacaaggaggacatgcgatcaagcttcaggtcgaatgtgttgacggggtcggctccccagcaggactacgataagctgaaggcactcggaggggaaaactccagcaaaactggactctctacgtcaggcaatgtggagaaaaacaaagctgttaagagagaaacagaagccagttctataaacctgagtgtttatgaaccttttaaagtcagaaaagcagaggataaattgaaggaaagctctgacaaggtgctggaaaacagagtcctagatgggaagctgagctccgagaagaatgacaccagcctccccagcgttgcgccatcaaagacaaagtcgtcctccaagctctcgtcctgcatcgctgccatcgcggctctcagcgctaaaaaggcggcttcagactcctgcaaagaaccagtggccaattcgagggaatcctccccgttaccaaaagaagtaaatgacagtccgagagccgctgacaagtctcctgaatcccagaatctcatcgacgggaccaaaaaaccatccctgaagcaaccggatagtcccagaagcatctcaagtgagaacagcagcaaaggatccccgtcctctcccgcagggtccacaccagcaatccccaaagtccgcataaaaaccattaagacatcttctggggaaatcaagagaacagtgaccagggtattgccagaagtggatcttgactctggaaagaaaccttccgagcagacagcgtccgtgatggcctctgtgacatcccttctgtcgtctccagcatcagccgccgtcctttcctctccccccagggcgcctctccagtctgcggtcgtgaccaatgcagtttcccctgcagagctcacccccaaacaggtcacaatcaagcctgtggctactgctttcctcccagtgtctgctgtgaagacggcaggatcccaagtcattaatttgaagctcgctaacaacaccacggtgaaagccacggtcatatctgctgcctctgtccagagtgccagcagcgccatcattaaagctgccaacgccatccagcagcaaactgtcgtggtgccggcatccagcctggccaatgccaaactcgtgccaaagactgtgcaccttgccaaccttaaccttttgcctcagggtgcccaggccacctctgaactccgccaagtgctaaccaaacctcagcaacaaataaagcaggcaataatcaatgcagcagcctcgcaaccccccaaaaaggtgtctcgagtccaggtggtgtcgtccttgcagagttctgtggtggaagctttcaacaaggtgctgagcagtgtcaatccagtccctgtttacatcccaaacctcagtcctcccgccaatgcagggatcacgttaccgacgcgtgggtacaagtgcttggagtgtggggactcctttgcacttgaaaagagtctgacccagcactacgacagacggagcgtgcgcatcgaagtaacgtgcaaccattgtacaaagaacctcgttttttacaacaaatgcagcctcctttcccatgcccgtgggcataaggagaaaggggtggtaatgcaatgctcccacttaattttaaagccagtcccagcagatcaaatgatagtttctccgtcaagcaatacttccacttcaacttccactcttcagagccctgtgggagctggcacacacactgtcacaaaaattcagtctggcataactgggacagtcatatcggctccttcaagcactcccatcaccccagccatgcccctagatgaagacccctccaaactgtgtagacatagtctaaaatgtttggagtgtaatgaagtcttccaggacgagacatcactggctacacatttccagcaggctgcagatacctgctcatatcctttgagacaagctctctgggctgacctaaaggaatgtgaggtgctacaccccgtcaaagcatctgcattgccgggaccggatatgagcatgaaacctagtgccgccctgtctccatcccctgcacttccctttctcccaccaacttctgacccaccagaccacccacccagggagccacctccacagcccatcatgccttcagtattctctccagacaaccctctgatgctctctgctttccccagctcactgttggtgacaggggacgggggcccttgcctcagtggggctggggctggcaaggtcattgtcaaagtcaagacagaaggggggtcagctgagccctctcaaactcagaactttatccttactcagactgccctcaattcgactgccccgggcactccctgtggaggccttgagggtcctgcacctccatttgtgacagcatctaatgtgaagaccattctgccctctaaggctgttggtgtcagccaggagggtcctccaggccttccgcctcagcctccaccaccagttgctcaactggtccccattgtgcccctggaaaaagcttggccagggccacatgggacaaccggggaaggaggtcctgtggccactctatccaagccttccctaggtgaccgctccaaaatttccaaggacgtttatgagaacttccgtcagtggcagcgttacaaagccttggcccggaggcacctatcccagagtcctgacacagaagctctttcctgttttcttatcccagtgcttcgttccctggcccggctgaagcccactatgaccctggaggagggactgccattggctgtgcaggagtgggagcacaccagcaactttgaccggatgatcttttatgagatggcagaaaggttcatggagtttgaggctgaggagatgcagattcagaacacacagctgatgaatgggtctcagggcctgtctcctgcaacccctttgaaacttgatcctctagggcccctggcctctgaggtttgccagcagccagtgtacattccgaagaaggcagcctccaagacacgggccccccgccggcgtcagcgtaaagcccagagacctcctgctcctgaggcacccaaggagatcccaccagaagctgtgaaggagtatgttgacatcatggaatggctggtggggactcacttggccactggggagtcagatggaaaacaagaggaagaagggcagcagcaggaggaggaagggatgtatccagatccaggtctcctgagctacatcaatgagctgtgttctcagaaggtctttgtctccaaggtggaggctgtcattcaccctcaatttctggcagatctgctgtccccagaaaaacagagagatcccttggccttaattgaggagctagagcaagaagaaggactcactcttgcccagctggtccagaagcgactcatggccttggaagaggaggaagatgcagaggcgcctccaagtttcagtggcgctcagttggactcaagtccttctggttctgttgaggatgaagatggggatgggcggcttcggccctcacctgggcttcagggggctgggggcgccgcttgccttggaaaggtttcttcttcaggaaaacgggcaagagaagtgcatggtgggcaggagcaagccctagatagccccagagggatgcacagggatgggaacactctgccatcccccagcagctgggacctgcagccagaacttgcagctccacagggaactccgggacccttgggtgtggagaggagagggtctgggaaggttataaaccaggtatctctacatcaggatggccatctaggaggcgctgggcctcctgggcactgcctggtggctgataggacttcagaggctctgcccctttgttggcagggaggcttccagcctgagagcactcccagtttggatgctggacttgcagagctggctcctctgcaaggacaagggttagaaaagcaagtcctgggattgcagaaaggacaacaaacagggggtcgtggagtgcttcctcaagggaaggagcctttagcagtgccctgggaaggctcttcaggagccatgtggggagatgacagaggtacccccatggctcagagttatgatcagaatccttcccctagagcagctggggagagggacgatgtctgtctcagcccaggagtttggctgagcagtgagatggatgctgtaggcttggagctgcctgtacaaatagaggaggtcatagagagcttccaagttgagaagtgtgtaactgagtatcaggaaggctgccagggactgggctccaggggcaacatttccctgggtcctggagaaaccctagtacctggggatacggagagcagtgtgattccctgtggaggcacagttgcggcagctgccctagaaaagagaaactattgcagcttgccaggacctttgagggccaacagcccacccttgaggtccaaagaaaatcaagaacagagctgtgaaaccgtagggcatcccagtgatctgtgggcagaaggttgcttcccattgctagaaagtggtgattccacactggggtcttccaaagaaacccttccacccacatgccaaggcaatctccttatcatggggactgaggatgcctcctccttgcctgaagccagtcaagaggcagggagcagaggcaattccttttctcctctgttggaaaccatagaacctgtcaacatactagatgttaaagatgactgtggcctccaactaagggtcagcgaggacacctgcccactgaatgttcattcttatgacccccaaggagaaggcagggtggatcctgatctgtccaagcctaaaaaccttgctcctttacaagagagtcaggagtcttacacaactgggactcccaaagcaacatcttctcaccagggccttggaagcactttgcctagaaggggaaccaggaatgccatagttccgagagaaacttctgttagtaaaacacacaggtcagcagacagggccaaaggaaaggagaaaaagaaaaaggaagcagaggaagaggatgaggaactctccaactttgcttacctcttggcctctaaacttagcctctcaccaagggagcatcccctcagtcctcaccatgcctcaggaggtcagggcagccagagagcatcccacctgctccctgctggagcaaaaggccccagcaaacttccatatcctgttgccaagtctgggaagcgagctctagctggaggtccagcccctactgaaaagacaccccactcaggagctcaacttggggtccccagggagaaacccctagctctgggagtagttcgaccctcacagcctcgtaaaaggcggtgtgacagttttgtcacgggcagaaggaagaaacgacgtcgtagccagtagggagcagcgggaccatctgaccccacttgccagtccctaaaggtgggtgccccagagtagattccacccctgctgcccaccaatggagaatcccaatgttgaatctcatcccaatgttgttttgttgttctgcaaaagtggcaagcatggagagagaggtcagactggctaggctgcagggggaattacctttggaaggagctatatagaaaaaaaatgaataaagtgttttgttggaaaatgctc

SEQ ID NO: 4 - protein, 1929 aa, Artificial Sequence
  Description of Artificial Sequence Synthetic polypeptide
MTMGDMKTPDFDDLLAAFDIPDMVDPKAAIESGHDDHESHMKQNAHGEDDSHAPSSSDVGVSVIVKNVRNIDSSEGGEKDGHNPTGNGLHNGFLTASSLDSYSKDGAKSLKGDVPASEVTLKDSTFSQFSPISSAEEFDDDEKIEVDDPPDKEDMRSSFRSNVLTGSAPQQDYDKLKALGGENSSKTGLSTSGNVEKNKAVKRETEASSINLSVYEPFKVRKAEDKLKESSDKVLENRVLDGKLSSEKNDTSLPSVAPSKTKSSSKLSSCIAAIAALSAKKAASDSCKEPVANSRESSPLPKEVNDSPRAADKSPESQNLIDGTKKPSLKQPDSPRSISSENSSKGSPSSPAGSTPAIPKVRIKTIKTSSGEIKRTVTRVLPEVDLDSGKKPSEQTASVMASVTSLLSSPASAAVLSSPPRAPLQSAVVTNAVSPAELTPKQVTIKPVATAFLPVSAVKTAGSQVINLKLANNTTVKATVISAASVQSASSAIIKAANAIQQQTVVVPASSLANAKLVPKTVHLANLNLLPQGAQATSELRQVLTKPQQQIKQAIINAAASQPPKKVSRVQVVSSLQSSVVEAFNKVLSSVNPVPVYIPNLSPPANAGITLPTRGYKCLECGSDSFALEKSLTQHYDRRVRIEVTCNHCTKNLVFYNKCSLLSHARGHKEKGVVMQCSHLILKPVPADQMIVSPSSNTSTSTSTLQSPVGAGTHTVTKIQSGITGTVISAPSSTPITPAMPLDEDPSKLCRHSLKCLECNEVFQDETSLATHFQQAADTCSYPLRQALWADLKECEVLHPVKASALPGPDMSMKPSAALSPSPALPFLPPTSDPPDHPPREPPPQPIMPSVFSPDNPLMLSAFPSSLLVTGDGGPCLSGAGAGKVIVKVKTEGGSAEPSQTQNFILTQTALNSTAPGTPCGGLEGPAPPFVTASNVKTILPSKAVGVSQEGPPGLPPQPPPPVAQLVPIVPLEKAWPGPHGTTGEGGPVATLSKPSLGDRSKISKDVYENFRQWQRYKALARRHLSQSPDTEALSCFLIPVLRSLARLKPTMTLEEGLPLAVQEWEHTSNFDRMIFYEMAERFMEFEAEEMQIQNTQLMNGSQGLSPATPLKLDPLGPLASEVCQQPVYIPKKAASKTRAPRRRQRKAQRPPAPEAPKEIPPEAVKEYVDIMEWLVGTHLATGESDGKQEEEGQQQEEEGMYPDPGLLSYINELCSQKVFVSKVEAVIHPQFLADLLSPEKQRDPLALIEELEQEEGLTLAQLVQKRLMALEEEEDAEAPPSFSGAQLDSSPSGSVEDEDGDGRLRPSPGLQGAGGAACLGKVSSSGKRAREVHGGQEQALDSPRGMHRDGNTLPSPSSWDLQPELAAPQGTPGPLGVERRGSGKVINQVSLHQDGHLGGAGPPGHCLVADRTSEALPLCWQGGFQPESTPSLDAGLAELAPLQGQGLEKQVLGLQKGQQTGGRGVLPQGKEPLAVPWEGSSGAMWGDDRGTPMAQSYDQNPSPRAAGERDDVCLSPGVWLSSEMDAVGLELPVQIEEVIESFQVEKCVTEYQEGCQGLGSRGNISLGPGETLVPGDTESSVIPCGGTVAAAALEKRNYCSLPGPLRANSPPLRSKENQEQSCETVGHPSDLWAEGCFPLLESGDSTLGSSKETLPPTCQGNLLIMGTEDASSLPEASQEAGSRGNSFSPLLETIEPVNILDVKDDCGLQLRVSEDTCPLNVHSYDPQGEGRVDPDLSKPKNLAPLQESQESYTTGTPKATSSHQGLGSTLPRRGTRNAIVPRETSVSKTHRSADRAKGKEKKKKEAEEEDEELSNFAYLLASKLSLSPREHPLSPHHASGGQGSQRASHLLPAGAKGPSKLPYPVAKSGKRALAGGPAPTEKTPHSGAQLGVPREKPLALGVVRPSQPRKRRCDSFVTGRRKKRRRSQ

SEQ ID NO: 5 - protein, 50 aa, Homo sapiens
PAGSTPAIPKVRIKTIKTSSGEIKRTVTRVLPEVDLDSGKKPSEQTASVM

SEQ ID NO: 6 - DNA, 135 bp, Artificial Sequence
  Description of Artificial Sequence Synthetic polynucleotide
aggacgagacatcactggctacacatttccagcaggctgcagatacctgctcatatcctttgagacaagctctctgggctgacctaaaggaatgtgaggtgctacaccccgtcaaagcatctgcattgccgggac

SEQ ID NO: 7 - DNA, 5790 bp, Artificial Sequence
  Description of Artificial Sequence Synthetic polynucleotide
[391 bp omitted]cgatctccagtgctgaagagtttgatgacgacgagaagattgaggtggatgacccccctgacaaggaggacatgcgatcaagcttcaggtcgaatgtgttgacggggtcggctccccagcaggactacgataagctgaaggcactcggaggggaaaactccagcaaaactggactctctacgtcaggcaatgtggagaaaaacaaagctgttaagagagaaacagaagccagttctataaacctgagtgtttatgaaccttttaaagtcagaaaagcagaggataaattgaaggaaagctctgacaaggtgctggaaaacagagtcctagatgggaagctgagctccgagaagaatgacaccagcctccccagcgttgcgccatcaaagacaaagtcgtcctccaagctctcgtcctgcatcgctgccatcgcggctctcagcgctaaaaaggcggcttcagactcctgcaaagaaccagtggccaattcgagggaatcctccccgttaccaaaagaagtaaatgacagtccgagagccgctgacaagtctcctgaatcccagaatctcatcgacgggaccaaaaaaccatccctgaagcaaccggatagtcccagaagcatctcaagtgagaacagcagcaaaggatccccgtcctctcccgcagggtccacaccagcaatccccaaagtccgcataaaaaccattaagacatcttctggggaaatcaagagaacagtgaccagggtattgccagaagtggatcttgactctggaaagaaaccttccgagcagacagcgtccgtgatggcctctgtgacatcccttctgtcgtctccagcatcagccgccgtcctttcctctccccccagggcgcctctccagtctgcggtcgtgaccaatgcagtttcccctgcagagctcacccccaaacaggtcacaatcaagcctgtggctactgctttcctcccagtgtctgctgtgaagacggcaggatcccaagtcattaatttgaagctcgctaacaacaccacggtgaaagccacggtcatatctgctgcctctgtccagagtgccagcagcgccatcattaaagctgccaacgccatccagcagcaaactgtcgtggtgccggcatccagcctggccaatgccaaactcgtgccaaagactgtgcaccttgccaaccttaaccttttgcctcagggtgcccaggccacctctgaactccgccaagtgctaaccaaacctcagcaacaaataaagcaggcaataatcaatgcagcagcctcgcaaccccccaaaaaggtgtctcgagtccaggtggtgtcgtccttgcagagttctgtggtggaagctttcaacaaggtgctgagcagtgtcaatccagtccctgtttacatcccaaacctcagtcctcccgccaatgcagggatcacgttaccgacgcgtgggtacaagtgcttggagtgtggggactcctttgcacttgaaaagagtctgacccagcactacgacagacggagcgtgcgcatcgaagtaacgtgcaaccattgtacaaagaacctcgttttttacaacaaatgcagcctcctttcccatgcccgtgggcataaggagaaaggggtggtaatgcaatgctcccacttaattttaaagccagtcccagcagatcaaatgatagtttctccgtcaagcaatacttccacttcaacttccactcttcagagccctgtgggagctggcacacacactgtcacaaaaattcagtctggcataactgggacagtcatatcggctccttcaagcactcccatcaccccagccatgcccctagatgaagacccctccaaactgtgtagacatagtctaaaatgtttggagtgtaatgaagtcttccaggacgagacatcactggctacacatttccagcaggctgcagatacctgctcatatcctttgagacaagctctctgggctgacctaaaggaatgtgaggtgctacaccccgtcaaagcatctgcattgccgggaccggatatgagcatgaaacctagtgccgccctgtctccatcccctgcacttccctttctcccaccaacttctgacccaccagaccacccacccagggagccacctccacagcccatcatgccttcagtattctctccagacaaccctctgatgctctctgctttccccagctcactgttggtgacaggggacgggggcccttgcctcagtggggctggggctggcaaggtcattgtcaaagtcaagacagaaggggggtcagctgagccctctcaaactcagaactttatccttactcagactgccctcaattcgactgccccgggcactccctgtggaggccttgagggtcctgcacctccatttgtgacagcatctaatgtgaagaccattctgccctctaaggctgttggtgtcagccaggagggtcctccaggccttccgcctcagcctccaccaccagttgctcaactggtccccattgtgcccctggaaaaagcttggccagggccacatgggacaaccggggaaggaggtcctgtggccactctatccaagccttccctaggtgaccgctccaaaatttccaaggacgtttatgagaacttccgtcagtggcagcgttacaaagccttggcccggaggcacctatcccagagtcctgacacagaagctctttcctgttttcttatcccagtgcttcgttccctggcccggctgaagcccactatgaccctggaggagggactgccattggctgtgcaggagtgggagcacaccagcaactttgaccggatgatcttttatgagatggcagaaaggttcatggagtttgaggctgaggagatgcagattcagaacacacagctgatgaatgggtctcagggcctgtctcctgcaacccctttgaaacttgatcctctagggcccctggcctctgaggtttgccagcagccagtgtacattccgaagaaggcagcctccaagacacgggccccccgccggcgtcagcgtaaagcccagagacctcctgctcctgaggcacccaaggagatcccaccagaagctgtgaaggagtatgttgacatcatggaatggctggtggggactcacttggccactggggagtcagatggaaaacaagaggaagaagggcagcagcaggaggaggaagggatgtatccagatccaggtctcctgagctacatcaatgagctgtgttctcagaaggtctttgtctccaaggtggaggctgtcattcaccctcaatttctggcagatctgctgtccccagaaaaacagagagatcccttggccttaattgaggagctagagcaagaagaaggactcactcttgcccagctggtccagaagcgactcatggccttggaagaggaggaagatgcagaggcgcctccaagtttcagtggcgctcagttggactcaagtccttctggttctgttgaggatgaagatggggatgggcggcttcggccctcacctgggcttcagggggctgggggcgccgcttgccttggaaaggtttcttcttcaggaaaacgggcaagagaagtgcatggtgggcaggagcaagccctagatagccccagagggatgcacagggatgggaacactctgccatcccccagcagctgggacctgcagccagaacttgcagctccacagggaactccgggacccttgggtgtggagaggagagggtctgggaaggttataaaccaggtatctctacatcaggatggccatctaggaggcgctgggcctcctgggcactgcctggtggctgataggacttcagaggctctgcccctttgttggcagggaggcttccagcctgagagcactcccagtttggatgctggacttgcagagctggctcctctgcaaggacaagggttagaaaagcaagtcctgggattgcagaaaggacaacaaacagggggtcgtggagtgcttcctcaagggaaggagcctttagcagtgccctgggaaggctcttcaggagccatgtggggagatgacagaggtacccccatggctcagagttatgatcagaatccttcccctagagcagctggggagagggacgatgtctgtctcagcccaggagtttggctgagcagtgagatggatgctgtaggcttggagctgcctgtacaaatagaggaggtcatagagagcttccaagttgagaagtgtgtaactgagtatcaggaaggctgccagggactgggctccaggggcaacatttccctgggtcctggagaaaccctagtacctggggatacggagagcagtgtgattccctgtggaggcacagttgcggcagctgccctagaaaagagaaactattgcagcttgccaggacctttgagggccaacagcccacccttgaggtccaaagaaaatcaagaacagagctgtgaaaccgtagggcatcccagtgatctgtgggcagaaggttgcttcccattgctagaaagtggtgattccacactggggtcttccaaagaaacccttccacccacatgccaaggcaatctccttatcatggggactgaggatgcctcctccttgcctgaagccagtcaagaggcagggagcagaggcaattccttttctcctctgttggaaaccatagaacctgtcaacatactagatgttaaagatgactgtggcctccaactaagggtcagcgaggacacctgcccactgaatgttcattcttatgacccccaaggagaaggcagggtggatcctgatctgtccaagcctaaaaaccttgctcctttacaagagagtcaggagtcttacacaactgggactcccaaagcaacatcttctcaccagggccttggaagcactttgcctagaaggggaaccaggaatgccatagttccgagagaaacttctgttagtaaaacacacaggtcagcagacagggccaaaggaaaggagaaaaagaaaaaggaagcagaggaagaggatgaggaactctccaactttgcttacctcttggcctctaaacttagcctctcaccaagggagcatcccctcagtcctcaccatgcctcaggaggtcagggcagccagagagcatcccacctgctccctgctggagcaaaaggccccagcaaacttccatatcctgttgccaagtctgggaagcgagctctagctggaggtccagcccctactgaaaagacaccccactcaggagctcaacttggggtccccagggagaaacccctagctctgggagtagttcgaccctcacagcctcgtaaaaggcggtgtgacagttttgtcacgggcagaaggaagaaacgacgtcgtagccagtag

SEQ ID NO: 8 - protein, 1929 aa, Artificial Sequence
  Description of Artificial Sequence Synthetic polypeptide
MTMGDMKTPDFDDLLAAFDIPDMVDPKAAIESGHDDHESHMKQNAHGEDDSHAPSSSDVGVSVIVKNVRNIDSSEGGEKDGHNPTGNGLHNGFLTASSLDSYSKDGAKSLKGDVPASEVTLKDSTFSQFSPISSAEEFDDDEKIEVDDPPDKEDMRSSFRSNVLTGSAPQQDYDKLKALGGENSSKTGLSTSGNVEKNKAVKRETEASSINLSVYEPFKVRKAEDKLKESSDKVLENRVLDGKLSSEKNDTSLPSVAPSKTKSSSKLSSCIAAIAALSAKKAASDSCKEPVANSRESSPLPKEVNDSPRAADKSPESQNLIDGTKKPSLKQPDSPRSISSENSSKGSPSSPAGSTPAIPKVRIKTIKTSSGEIKRTVTRVLPEVDLDSGKKPSEQTASVMASVTSLLSSPASAAVLSSPPRAPLQSAVVTNAVSPAELTPKQVTIKPVATAFLPVSAVKTAGSQVINLKLANNTTVKATVISAASVQSASSAIIKAANAIQQQTVVVPASSLANAKLVPKTVHLANLNLLPQGAQATSELRQVLTKPQQQIKQAIINAAASQPPKKVSRVQVVSSLQSSVVEAFNKVLSSVNPVPVYIPNLSPPANAGITLPTRGYKCLECGDSFALEKSLTQHYDRRSVRIEVTCNHCTKNLVFYNKCSLLSHARGHKEKGVVMQCSHLILKPVPADQMIVSPSSNTSTSTSTLQSPVGAGTHTVTKIQSGITGTVISAPSSTPITPAMPLDEDPSKLCRHSLKCLECNEVFQDETSLATHFQQAADTCSYPLRQALWADLKECEVLHPVKASALPGPDMSMKPSAALSPSPALPFLPPTSDPPDHPPREPPPQPIMPSVFSPDNPLMLSAFPSSLLVTGDGGPCLSGAGAGKVIVKVKTEGGSAEPSQTQNFILTQTALNSTAPGTPCGGLEGPAPPFVTASNVKTILPSKAVGVSQEGPPGLPPQPPPPVAQLVPIVPLEKAWPGPHGTTGEGGPVATLSKPSLGDRSKISKDVYENFRQWQRYKALARRHLSQSPDTEALSCFLIPVLRSLARLKPTMTLEEGLPLAVQEWEHTSNFDRMIFYEMAERFMEFEAEEMQIQNTQLMNGSQGLSPATPLKLDPLGPLASEVCQQPVYIPKKAASKTRAPRRRQRKAQRPPAPEAPKEIPPEAVKEYVDIMEWLVGTHLATGESDGKQEEEGQQQEEEGMYPDPGLLSYINELCSQKVFVSKVEAVIHPQFLADLLSPEKQRDPLALIEELEQEEGLTLAQLVQKRLMALEEEEDAEAPPSFSGAQLDSSPSGSVEDEDGDGRLRPSPGLQGAGGAACLGKVSSSGKRAREVHGGQEQALDSPRGMHRDGNTLPSPSSWDLQPELAAPQGTPGPLGVERRGSGKVINQVSLHQDGHLGGAGPPGHCLVADRTSEALPLCWQGGFQPESTPSLDAGLAELAPLQGQGLEKQVLGLQKGQQTGGRGVLPQGKEPLAVPWEGSSGAMWGDDRGTPMAQSYDQNPSPRAAGERDDVCLSPGVWLSSEMDAVGLELPVQIEEVIESFQVEKCVTEYQEGCQGLGSRGNISLGPGETLVPGDTESSVIPCGGTVAAAALEKRNYCSLPGPLRANSPPLRSKENQEQSCETVGHPSDLWAEGCFPLLESGDSTLGSSKETLPPTCQGNLLIMGTEDASSLPEASQEAGSRGNSFSPLLETIEPVNILDVKDDCGLQLRVSEDTCPLNVHSYDPQGEGRVDPDLSKPKNLAPLQESQESYTTGTPKATSSHQGLGSTLPRRGTRNAIVPRETSVSKTHRSADRAKGKEKKKKEAEEEDEELSNFAYLLASKLSLSPREHPLSPHHASGGQGSQRASHLLPAGAKGPSKLPYPVAKSGKRALAGGPAPTEKTPHSGAQLGVPREKPLALGVVRPSQPRKRRCDSFVTGRRKKRRRSQ

(19) United States
(12) Patent Application Publication
(10) Pub. No.: US 2018/0346988 A1
(43) Pub. Date: Dec. 6, 2018

(54) ZNF532 FOR DIAGNOSIS AND TREATMENT OF CANCER

(71) Applicant: THE BRIGHAM AND WOMEN'S HOSPITAL, INC., Boston, MA (US)

(72) Inventors: Christopher Alexander French, Boston, MA (US); Mitzi I. Kuroda, Brookline, MA (US); Erica M. Walsh Michel, Somerville, MA (US); Artyom A. Alekseyenko, Brookline, MA (US)

(73) Assignee: THE BRIGHAM AND WOMEN'S HOSPITAL, INC., Boston, MA (US)

(21) Appl. No.: 15/571,359

(22) PCT Filed: May 4, 2016
(86) PCT No.: PCT/US16/30674

§ 371 Date: Nov. 2, 2017

Related U.S. Application Data
(60) Provisional application No. 62/156,497, filed on May 4, 2015.

(51) Int. Cl. C12Q 1/6886

(52) U.S. Cl. CPC C12Q 1/6886; C12Q 2600/158; C12Q 2600/106

(57) ABSTRACT

The present invention relates to the use of ZNF532 inhibitors for the treatment of cancer, in particular cancer with cells that express the ZNF532 protein or harbor the ZNF532-NUT fusion gene. In some embodiments, the cancer is selected from the group consisting of NUT midline carcinoma (NMC), Ewing sarcoma, and head and neck squamous cell carcinoma.

### CROSS-REFERENCE TO RELATED APPLICATION

This application claims benefit under 35 U.S.C. § 119(e) of U.S. Provisional Application No. 62/156,497, filed May 4, 2015, the contents of which are incorporated herein by reference in their entireties.

### GOVERNMENT SUPPORT

This invention was made with government support under grant Nos. 2R01CA124633-06A and T32HL007627-27 awarded by the National Institutes of Health. The government has certain rights in the invention.

### TECHNICAL FIELD

The present invention relates to cancer diagnosis and treatment.

### SEQUENCE LISTING

The instant application contains a Sequence Listing which has been submitted electronically in ASCII format and is hereby incorporated by reference in its entirety. Said ASCII copy, created on May 3, 2016, is named 043214-084971-PCT_SL.txt and is 70,653 bytes in size.

### BACKGROUND

NUT midline carcinoma (NMC) is a rare, aggressive, and incurable type of squamous cell carcinoma primarily affecting patients such as children and young adults. NMC is genetically defined by rearrangement of the NUT gene, resulting in fusion by chromosomal translocation to bromodomain-containing BET-family members (BRD4, BRD3), which encode BRD-NUT oncoproteins. NMC is very aggressive with a median survival of 6.7 months, and is unresponsive to a variety of chemotherapeutic strategies.

The oncogenic mechanism of BRD4-NUT is to block differentiation of NMC cells (FIG. 1), and this is dependent upon the binding of BRD4's dual bromodomains to chromatin. Inhibition of this interaction with a new class of small molecule inhibitors, bromodomain inhibitors (BET inhibitor) (Filippakopoulos P, et al., Nature. 2010; 468(7327):1067-73; Stathis A, et al., Cancer Discovery. 2016 in press.), is being used as targeted therapy of NMC, with at least three clinical trials recently opened. Toxicity and resistance to BET inhibitors has already been seen in NMC and other cancers. Thus, a better understanding of the oncogenic mechanism of BRD4-NUT to block differentiation is needed to identify additional therapeutics in this disease.

### SUMMARY

As described herein, the inventors have discovered the expression of a novel zinc finger protein that interacts with BRD4-NUT on chromatin, ZNF532. Expression of ZNF532 protein is higher in certain cancers (e.g., NMC, Ewing sarcoma, or head and neck squamous cell carcinoma) than in normal tissues, where the level of ZNF532 protein is low or absent. Expression of ZNF532 is required for blockade of differentiation, maintenance of proliferation, and invasiveness of several head and neck squamous cell carcinoma (HNSQC) cell lines tested. Additionally, the inventors have discovered a novel ZNF532-NUT fusion gene in a patient with NMC, underscoring the importance of ZNF532 in NMC pathogenesis. Furthermore, the inventors have discovered that ZNF532-NUT is required for the blockage of differentiation and maintenance of proliferation of ZNF532-NUT+NMCs, in a BRD4-dependent manner. And thus ZNF532 is a therapeutic target in cancer that expresses ZNF532 or harbors the ZNF532-NUT fusion gene. As toxicity and resistance to BET inhibitors has already been seen in NMC and other cancers, the invention described herein also permits one to select subjects in whom a BET inhibitor therapy can be effective.

In one aspect, the invention relates to a method of treating cancer in a subject comprising administering a pharmaceutically-effective amount of a ZNF532 inhibitor to the subject. In some embodiments, the cancer expresses ZNF532 protein or comprises a ZNF532-NUT fusion gene.

In one aspect, the invention relates to a method of treating cancer in a subject comprising requesting a test to measure a level of ZNF532 in a sample obtained from the subject, and administering to the subject a pharmaceutically-effective amount of a ZNF532 inhibitor and/or a BET inhibitor in response to the level of ZNF532 above a reference level.

In another aspect, the invention relates to a method of treating cancer in a subject comprising requesting a test to determine whether a ZNF532-NUT fusion gene is present in a sample obtained from the subject, and administering to the subject a pharmaceutically-effective amount of a ZNF532 inhibitor and/or a BET inhibitor in response to the ZNF532-NUT fusion gene identified to be present in the sample.

In some embodiments of any one of the foregoing aspects, the cancer is selected from the group consisting of NUT midline carcinoma (NMC), Ewing sarcoma, and head and neck squamous cell carcinoma.

In some embodiments of any one of the foregoing aspects, the ZNF532 inhibitor is selected from the group consisting of a small molecule, an antibody, an antibody fragment, RNAi, siRNA, a ZNF532 decoy molecule, a polypeptide that blocks the binding of ZNF532 with NUT and/or BRD4.

In some embodiments of any one of the foregoing aspects, the BET inhibitor is selected from the group consisting of: JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), GSK-525762A, LY294002, 1-[2-(1/-/-benzimidazol-2-ylthio)ethyl]-1,3-dihydro-3-methyl-2H-benzinidazole-2-thione, 1-methylethyl ((2S,4R)-1-acetyl-2-methyl-6-{4-[(methylamino)methyl]phenyl}-1,2,3,4-tetrahydro-4-quinolinyl)carbamate, 2-[(4S)-6-(4-Chlorophenyl)-1-methyl-8-(methyloxy)-4H-[1,2,4]triazolo [4,3-a][1,4]benzodiazepin-4-yl]-N-ethylacetamide, 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one, 7-(3,5-dimethyl-4-isoxazolyl)-8-(methoxy)-1-[(1R)-phenylethyl]-2-(tetrahydro-2H-pyran-4-yl)-1H-imidazo[4,5-c]quinolone, 4-{(2S,4R)-1-acetyl-4-[(4-chlorophenyl)amino]-2-methyl-1,2,3,4-tetrahydro-6-quinolinyl}benzoic acid, and N-{1-methyl-7-[4-(1-piperidinylmethyl)phenyl][1,2,4]triazolo[4,3-a]quinolin-4-yl}urea.

In some embodiments of any one of the foregoing aspects, the sample is a cancer sample.

In some embodiments of any one of the foregoing aspects, the subject is a mammal.

In some embodiments of any one of the foregoing aspects, the mammal is a human.

In yet another aspect, the invention relates to a method of determining whether a BET inhibitor therapy can be effective in a subject having cancer, the method comprising: measuring, in a sample obtained from the subject, a level of ZNF532; and determining that the therapy can be effective if the level of ZNF532 is above a reference level. In some embodiments, the method further comprises administering the BET inhibitor therapy to the subject when the level of ZNF532 is above the reference level.

In another aspect, the invention relates to a method of determining whether a BET inhibitor therapy can be effective in a subject having cancer, the method comprising: determining, in a sample obtained from the subject, whether a ZNF532-NUT fusion gene is present in the sample; and determining that the therapy can be effective if the ZNF532-NUT fusion gene is present in the sample. In some embodiments, the method further comprises administering the BET inhibitor therapy to the subject when the ZNF532-NUT fusion gene is present in the sample.

### BRIEF DESCRIPTION OF THE DRAWINGS

FIG. 1 is a set of microscope graphs demonstrating that siRNA knockdown ofBRD4-NUT in 797NMC cells results in terminal squamous differentiation characterized by flattening, cytoplasmic and nuclear enlargement, open chromatin, and stratification.

FIGS. 2A-2C describe experimental data showing that wild type ZNF532 is required for the blockade of differentiation in NMC. (FIG. 2A) Endogenous wtZNF532 co-localizes with HA-tagged BRD4-NUT induced in 797TRex NMC cells. (FIG. 2B) Knockdown of ZNF532 induces differentiation of theTC-797 cells as determined by involucrin expression, a marker of epithelial differentiation (negative control is scrambled siRNA. (FIG. 2C) Quantitative RT-PCR to measure knockdown of ZNF532 in TC-797 cells at 72 h as in (FIG. 2B). ZNF532 A-C are different primer pairs to the ZNF532 coding sequence.

FIGS. 3A-3F are microscope graphs demonstrating that in vivo expression of wtZNF532 in NMC. wtZNF532 expression is seen in NMCs with all four translocation types (FIGS. 3A-3D), and in the one Ewing sarcoma tested (FIG. 3F). Note the speckled staining (FIG. 3A-3B) that matches the pattern of NUT staining in many NMCs. (FIG. 3D) ZNF532 staining, like that of BRD-NUT, is only present within the poorly differentiated component in areas of squamous differentiation. (FIG. 3E) wtZNF532 is undetectable in 293T cultured cells.

FIG. 4 shows ChIP-seq and nascent RNA-seq analysis of three different cell lines and one patient tissue (1015), reveal that ZNF532 locus is enriched by BRD4/BRD3-NUT. Transcription is seen in two of the NMC cell lines, 797 and 1015, and is shown to diminish in the presence of JQ1.

FIGS. 5A-5F describe experimental data showing ZNF532-NUT fusion in NMC. (FIG. 5A) Diagnosis of NMC in a 61 year old female with a pulmonary tumor. Immunohistochemistry demonstrates a speckled pattern of nuclear staining using the C52 antibody against NUT (CST). This finding is diagnostic of NUT midline carcinoma. (FIG. 5B) RNA-seq sequence of the 24335 line reveals ZNF532-NUT fusion transcript (SEQ ID NO: 6). ZNF532, NUTintronl, NUTexons2-7 (FIG. 5C) Karyotype taken from cell line, 24335, derived from this patient demonstrates 47XX+7, t(15; 18)(q14; q23). (FIG. 5D) RT-PCR using ZNF532 forward and NUT reverse primers in 24335 confirms the presence of a ZNF532-NUT fusion transcript in 24335. (FIG. 5E) Dual color fluorescent in situ hybridization (FISH) demonstrates split apart of ZNF532 (centromeric 5′, telomeric 3) and fusion of ZNF532 (chr.18q21.32) to NUT(chr.15q14). (FIG. 5F) Immunoblot stained with the C52 anti-NUT antibody demonstrates an aberrant NUT band in the 24335 cell line. The large size (>245 kDa) indicates the presence of a NUT-fusion protein that is close in size, but different in size to BRD4-NUT and BRD3-NUT proteins, shown.

FIGS. 6A-6B are predicted sequences of ZNF532-NUT. (FIG. 6A) Nucleotide sequence of ZNF532-NUT. ZNF532, NUT intronl, NUT exons 2-7 (SEQ ID NO: 7). This sequence has been confirmed by sequencing of PCR amplified cDNA from the 24335 cell line. (FIG. 6B) predicted amino acid sequence of ZNF532-NUT. ZNF532, NUT intronl, NUT exons 2-7 (SEQ ID NO: 8).

FIG. 7 is a schematic of NUT fusions with ZNF532 and BRD4.

FIGS. 8A-8B describe experimental data showing differentiation of ZNF532-NUT+24335 cells in response to NUT knockdown or BET inhibitor. (FIG. 8A) Knockdown of ZNF532-NUT (shNUT) induces differentiation of the same cell line as determined by keratin expression, a marker of epithelial differentiation (negative control is shC10 ORF). (FIG. 8B) Treatment of 24335 cells with the BET inhibitor, JQ1, results in differentiation of the cell line as determined by expression of involucrin, a marker of terminal squamous differentiation.

FIG. 9 is a model of NUT-fusion oncoproteins in action. Without wishing to be bound by theory, the model postulates that, through interaction with BRD4, a variety of NUT-fusion partners form similar oncogenic complexes to that of BRD4-NUT. DNA-sequence association specificity is conferred by ZNF532 in both ZNF532-NUT and BRD4-NUT complexes.

FIGS. 10A-10D demonstrate differentiation and arrested proliferation of ZNF532-NUT+24335 cells in response to NUT or ZNF532 knockdown, or BET inhibitor. (FIG. 10A) Immunoblot of involucrin expression, a marker of squamous differentiation. (FIG. 10B) H&E showing differentiation as evidenced by formation of cohesive spherical clusters, and marked accumulation of cytoplasm. (FIG. 10C) 500 nM JQ1 BET inhibitor was administered. Dose-response to JQ1 measured by cell titer glo. (FIG. 10D) Effects of ZNF532 and NUT knock down on number of mitoses quantified by immunohistochemistry using anti-histone H3phospho-serine antibody.

FIGS. 11A-11B shows regulation of MYC expression in a BET-dependent manner by ZNF532-NUT megadomain. (FIG. 11A) Localization of ZNF532-NUT and effects on transcription at the MYC locus based on ChIP-seq and RNA-seq. JQ1 was administered for 4 h prior to RNA collection. (FIG. 11B) Immunoblot demonstrates that NUT knock down results in decreased MYC protein in 24335 cells.

FIGS. 12A-12C show ZNF532 is required for the blockade of differentiation and proliferation in NMC. (FIG. 12A) Differentiation of PER-403NMC cells following knockdown of ZNF532. Photomicrographs of cells stained with Hemacolor Rapid Stain (EMD Millipore) demonstrating squamous differentiation (flattening, enlargement, opening of chromatin). (FIG. 12B) Differentiation of the PER-403 cells following ZNF532 knockdown, as determined by keratin expression, a marker of terminal squamous differentiation (negative control is scrambled siRNA). (FIG. 12C) Effects of ZNF532 knock down on number of mitoses in TC-797 AND per-403 NMC cells quantified by immunohistochemistry using anti-histone H3phospho-serine antibody.

FIG. 13 shows a model of how ZNF532 and MYC contribute to BRD4-NUT oncogenic function.

FIGS. 14A-14E ZNF532 is required for the blockade of differentiation, proliferation, and invasiveness of non-NMC head and neck squamous cancers (HNSQC). (FIG. 14A) Differentiation of four different HNSQC cell lines, but not control 293T cells, following knockdown of ZNF532. Photomicrographs of cells stained with Hemacolor Rapid Stain demonstrating squamous differentiation (flattening, nuclear and cytoplasmic enlargement, accumulation of cytoplasm, opening of chromatin). All photomicrographs are at same magnification (400×). (FIG. 14B) Differentiation of the HNSQC cells following ZNF532 knockdown, as determined by involucrin expression, a marker of terminal squamous differentiation (negative control is scrambled siRNA). (FIG. 14C) Effects of ZNF532 knockdown on invasiveness (matrigel invasion assay). (FIG. 14D) Proliferation scored by IHC for the mitosis marker, histone H3phospho-serine. (FIG. 14E) Immunohistochemistry using anti-ZNF532 demonstrating in vivo expression of wtZNF532 in one of two HNSQCs.

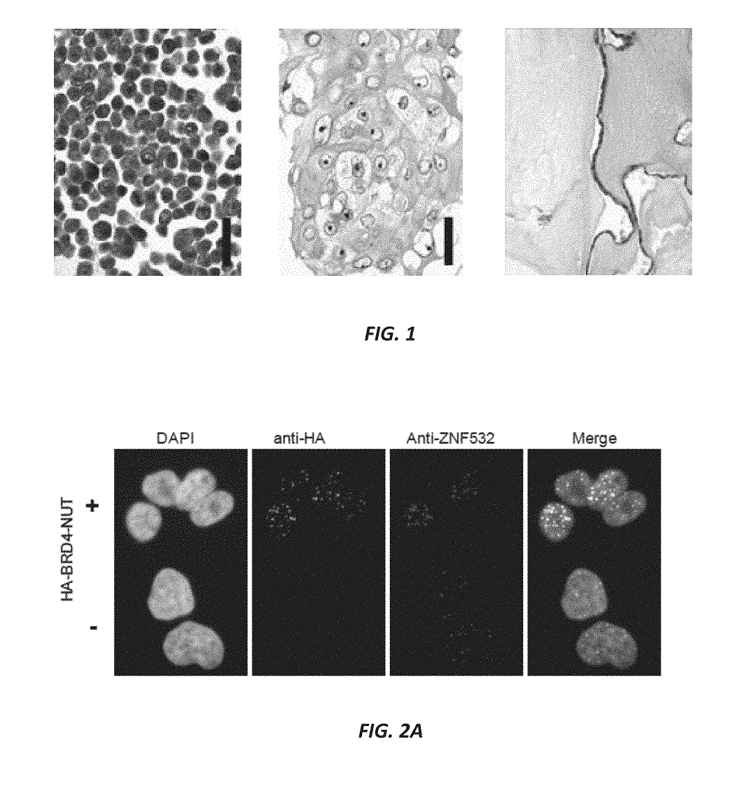

### DETAILED DESCRIPTION

The present invention is based, inter alia, on the discovery that ZNF532 is required for the blockade of differentiation and maintenance of proliferation in NMC and HNSQCs. In some embodiments, the NMC is characterized by the presence of a BRD4-NUT fusion gene (i.e., BRD4-NUT+NMC). In some embodiments, the NMC is characterized by the presence of a ZNF532-NUT fusion gene (i.e., ZNF532-NUT+NMC). In some embodiments, the NMC is characterized by the presence of a BRD3-NUT fusion gene (i.e., BRD3-NUT+NMC). In some embodiments, the NMC is characterized by the presence of a NSD3-NUT fusion gene (i.e., NSD3-NUT+NMC). In some embodiments, the HNSQC is characterized by expression of ZNF532. Accordingly, some aspects and embodiments of the present invention provide methods and compositions comprising ZNF532 inhibitors and/or BET inhibitors for the treatment of cancers.

ZNF532 is a protein that in humans is encoded by the ZNF532 gene. The human ZNF532 protein sequence has an accession NO. of NP_060651 (SEQ ID NO.: 1). The human ZNF532 mRNA sequence has an accession NO. of NM_018181 (SEQ ID NO.: 2).

SEQ ID NO.: 1 is shown as follows:

| | |
|---|---|
| 1 | mtmgdmktpd fddllaafdi pdmvdpkaai esghddhesh mkqnahgedd shapsssdvg |
| 61 | vsvivknvrn idsseggekd ghnptgnglh ngfltassld syskdgaksl kgdvpasevt |
| 121 | lkdstfsqfs pissaeefdd dekievddpp dkedmrssfr snvltgsapq qdydklkalg |
| 181 | genssktgls tsgnveknka vkreteassi nlsvyepfkv rkaedklkes sdkvlenrvl |
| 241 | dgklsseknd tslpsvapsk tksssklssc iaaiaalsak kaasdsckep vansresspl |
| 301 | pkevndspra adkspesqnl idgtkkpslk qpdsprsiss ensskgspss pagstpaipk |
| 361 | vriktiktss geikrtvtrv lpevdldsgk kpseqtasvm asvtsllssp asaavlsspp |
| 421 | raplqsavvt navspaeltp kqvtikpvat aflpvsavkt agsqvinlkl annttvkatv |
| 481 | isaasvqsas saiikaanai qqqtvvvpas slanaklvpk tvhlanlnll pqgaqatsel |
| 541 | rqvltkpqqq ikqaiinaaa sqppkkvsrv qvvsslqssv veafnkvlss vnpvpvyipn |
| 601 | lsppanagit lptrgykcle cgdsfaleks ltqhydrrsv rievtcnhct knlvfynkcs |
| 661 | llsharghke kgvvmqcshl ilkpvpadqm ivspssntst ststlqspvg agthtvtkiq |
| 721 | sgitgtvisa psstpitpam pldedpsklc rhslkclecn evfqdetsla thfqqaadts |
| 781 | gqktcticqm llpnqcsyas hqrihqhksp ytcpecgaic rsvhfqthvt knclhytrrv |
| 841 | gfrcvhcnvv ysdvaalksh iqgshcevfy kcpicpmafk sapsthshay tqhpgikige |
| 901 | pkiiykcsmc dtvftlqtll yrhfdqhien qkvsvfkcpd csllyaqkql mmdhiksmhg |
| 961 | tlksiegppn lginlplsik patqnsanqn kedtksmngk eklekkspsp vkksmetkkv |
| 1021 | aspgwtcwec dclfmqrdvy ishvrkehgk qmkkhperqc dksfssshsl crhnrikhkg |
| 1081 | irkvyacshc pdsrrtftkr lmlekhvqlm hgikdpdlke mtdatneeet eikedtkvps |
| 1141 | pkrkleepvl efrpprgait qplkklkinv fkvhkcavcg fttenllqfh ehipqhksdg |
| 1201 | ssyqcrecgl cytshvslsr hlfivhklke pqpvskqnga gednqqenkp shedespdga |
| 1261 | vsdrkckvca ktfeteaaln thmrthgmaf ikskrmssae k |

SEQ ID NO.: 2 is shown as follows:

| | |
|---|---|
| 1 | gccatgtttc aatctggccc cagtggcttt ttctctgaaa gcaaacgtgt gtcttttaca |
| 61 | ccagggcttt ctccccaccc cagggggtgt cttccatcct tttgtggctc agttgaaggc |
| 121 | gaaaagggct ccaaaccact aactaaccag aggagagccc cttcttccac ctccagggag |
| 181 | aatttcagat ttaatttgtc cgaagatagc gtgctctctt cttactcatt tgccatcatt |
| 241 | acgaggaaaa caaaccacca ccttggcttc aagatcctgg gtagaggctc acggtctttt |
| 301 | caaccatctt tggcgaggcc ttgcttcctt ccactcgagg tatgttctgt cttgtgcttt |
| 361 | ttcttttaga agctactaaa gggtgttggg gatgcttctg actattatga aggccaaaag |
| 421 | gcctgttgac tggggctgct tttaaccctt tcctatttgc tgagaatgca gccgtgtgac |
| 481 | agtaactgaa cattggtcta aagtctttcc aaaaggtcaa ggttcacaag aacatctgct |
| 541 | caaattaatg accatggggg atatgaagac cccagacttt gatgacctcc tggcagcatt |
| 601 | tgacatccca gatatggtcg atcctaaagc agctattgag tctggacacg atgaccatga |
| 661 | aagccacatg aagcagaatg ctcacggaga ggatgactcc cacgcaccat catcttctga |
| 721 | tgtgggtgtc agcgttatcg tcaagaatgt tcggaacatt gactcttccg agggcgggga |
| 781 | gaaagacggc cacaacccca ctggcaatgg cttacataat gggtttctca cagcatcctc |
| 841 | ccttgacagt tacagtaaag atggagcaaa gtccttgaaa ggagatgtgc ctgcctctga |
| 901 | ggtgacactg aaagactcga cattcagcca gtttagcccg atctccagtg ctgaagagtt |
| 961 | tgatgacgac gagaagattg aggtggatga cccccctgac aaggaggaca tgcgatcaag |
| 1021 | cttcaggtcg aatgtgttga cggggtcggc tccccagcag gactacgata agctgaaggc |
| 1081 | actcggaggg gaaaactcca gcaaaactgg actctctacg tcaggcaatg tggagaaaaa |
| 1141 | caaagctgtt aagagagaaa cagaagccag ttctataaac ctgagtgttt atgaaccttt |
| 1201 | taaagtcaga aaagcagagg ataaattgaa ggaaagctct gacaaggtgc tggaaaacag |
| 1261 | agtcctagat gggaagctga gctccgagaa gaatgacacc agcctcccca gcgttgcgcc |
| 1321 | atcaaagaca aagtcgtcct ccaagctctc gtcctgcatc gctgccatcg cggctctcag |
| 1381 | cgctaaaaag gcggcttcag actcctgcaa agaaccagtg gccaattcga gggaatcctc |
| 1441 | cccgttacca aaagaagtaa atgacagtcc gagagccgct gacaagtctc ctgaatccca |
| 1501 | gaatctcatc gacgggacca aaaaaccatc cctgaagcaa ccggatagtc ccagaagcat |
| 1561 | ctcaagtgag aacagcagca aaggatcccc gtcctctccc gcagggtcca caccagcaat |
| 1621 | ccccaaagtc cgcataaaaa ccattaagac atcttctggg gaaatcaaga gaacagtgac |
| 1681 | cagggtattg ccagaagtgg atcttgactc tggaaagaaa ccttccgagc agacagcgtc |
| 1741 | cgtgatggcc tctgtgacat cccttctgtc gtctccagca tcagccgccg tcctttcctc |
| 1801 | tccccccagg gcgcctctcc agtctgcggt cgtgaccaat gcagtttccc ctgcagagct |
| 1861 | cacccccaaa caggtcacaa tcaagcctgt ggctactgct ttcctcccag tgtctgctgt |
| 1921 | gaagacggca ggatcccaag tcattaattt gaagctcgct aacaacacca cggtgaaagc |
| 1981 | cacggtcata tctgctgcct ctgtccagag tgccagcagc gccatcatta aagctgccaa |
| 2041 | cgccatccag cagcaaactg tcgtggtgcc ggcatccagc ctggccaatg ccaaactcgt |
| 2101 | gccaaagact gtgcaccttg ccaaccttaa ccttttgcct cagggtgccc aggccacctc |
| 2161 | tgaactccgc caagtgctaa ccaaacctca gcaacaaata aagcaggcaa taatcaatgc |
| 2221 | agcagcctcg caacccccca aaaaggtgtc tcgagtccag gtggtgtcgt ccttgcagag |
| 2281 | ttctgtggtg gaagctttca acaaggtgct gagcagtgtc aatccagtcc ctgtttacat |
| 2341 | cccaaacctc agtcctcccg ccaatgcagg gatcacgtta ccgacgcgtg ggtacaagtg |
| 2401 | cttggagtgt ggggactcct ttgcacttga aaagagtctg acccagcact acgacagacg |
| 2461 | gagcgtgcgc atcgaagtaa cgtgcaacca ttgtacaaag aacctcgttt tttacaacaa |
| 2521 | atgcagcctc ctttcccatg cccgtgggca taaggagaaa ggggtggtaa tgcaatgctc |
| 2581 | ccacttaatt ttaaagccag tcccagcaga tcaaatgata gtttctccgt caagcaatac |
| 2641 | ttccacttca acttccactc ttcagagccc tgtgggagct ggcacacaca ctgtcacaaa |
| 2701 | aattcagtct ggcataactg ggacagtcat atcggctcct tcaagcactc ccatcacccc |
| 2761 | agccatgccc ctagatgaag acccctccaa actgtgtaga catagtctaa aatgtttgga |
| 2821 | gtgtaatgaa gtcttccagg acgagacatc actggctaca catttccagc aggctgcaga |
| 2881 | tacgagtgga caaaagactt gcactatctg ccagatgctg cttcctaacc agtgcagtta |
| 2941 | tgcatcacac cagagaatcc atcagcacaa atctccctac acctgccctg agtgtggggc |
| 3001 | catctgcagg tcggtgcact tccagaccca cgtcaccaag aactgtctgc actacacgag |
| 3061 | gagagttggt ttccgatgtg tgcattgcaa tgttgtgtac tctgatgtgg ctgctctgaa |
| 3121 | gtctcacatt caaggttctc actgtgaagt cttctacaag tgtcctattt gtccaatggc |
| 3181 | gtttaagtct gccccaagca cacattccca cgcctacaca cagcatcctg gcatcaagat |
| 3241 | aggagaacca aaaataatat ataagtgttc catgtgcgac actgtgttca ccctgcaaac |
| 3301 | cttgctgtat cgccactttg accaacacat tgaaaaccag aaggtgtctg ttttcaagtg |
| 3361 | tccagactgt tctcttttat atgcacagaa gcaacttatg atggaccata tcaagtctat |
| 3421 | gcatggaaca ttgaaaagta ttgaagggcc tccaaacttg ggtataaact tgcctttgag |
| 3481 | cattaagcct gcaactcaaa attcagcaaa tcagaacaaa gaggacacca aatccatgaa |
| 3541 | tgggaaagag aaattggaaa agaaatctcc atctcctgtg aaaaaatcaa tggaaaccaa |
| 3601 | gaaagtggcc agtcctgggt ggacgtgttg ggagtgtgac tgcctgttca tgcagagaga |
| 3661 | tgtgtacata tcccacgtga ggaaggagca cgggaagcaa atgaagaaac acccctgccg |
| 3721 | ccagtgtgac aagtctttca gctcgtccca cagcctgtgc cggcacaacc ggatcaagca |
| 3781 | caaaggcatc aggaaagtgt acgcctgctc gcactgccca gactccagac gtacctttac |
| 3841 | caaacgtttg atgctggaga agcacgtcca gctgatgcat ggcatcaagg accctgacct |
| 3901 | gaaagaaatg acagatgcca ccaatgagga ggaaacagaa ataaaagaag acactaaggt |
| 3961 | ccccagtccc aagcggaagt tggaagaacc agttctggag ttcaggcctc cccgaggagc |
| 4021 | aatcactcaa ccactgaaaa agctgaaaat caatgttttt aaggttcaca agtgtgccgt |
| 4081 | gtgtggcttc accaccgaaa acctgctgca attccacgaa cacatccctc agcacaaatc |
| 4141 | ggatggttct tcctaccagt gccgggagtg tggcctctgc tacacgtctc acgtctctct |
| 4201 | gtccaggcac ctcttcatcg tacacaagtt aaaggaacct cagccagtgt ccaagcaaaa |
| 4261 | tggggctggg gaagataacc aacaggagaa caaacccagc cacgaggatg aatcccctga |
| 4321 | tggcgccgtg tcagacagaa agtgcaaagt gtgcgcaaaa acttttgaaa ctgaagctgc |
| 4381 | cttaaatact cacatgcgga cacacggcat ggccttcatc aaatccaaaa ggatgagctc |
| 4441 | agccgagaaa tagccacaga tgctccatga ggaaaatccc tgtccacatt ggaataaaaa |
| 4501 | agacattttt gttacaaagt ttgcagtata atagagttaa cagtactgtc taggctgttg |
| 4561 | caatatattc tctttcaatg taccttcctt cacctcgtcg tatatatcct cgataagtat |
| 4621 | taaaacagta tttgagttta aaagagtttg tatatattta aatgaataac tttttatact |
| 4681 | ctttgttaca tgtttgtatc agtatttagt ggaaaaccat ttgagttgtt ttgggttaga |
| 4741 | atttttcttt ttgtactgtt tctttaaaac agagttctta gtaacagggg cagttcctga |
| 4801 | attcaaataa accattttgt atgtttggat tttgaatggg ttaactaatt acaggctaaa |
| 4861 | ataatgcctt ttttagtgtt tttaattttt agaattcact acataaattg taagtaattg |
| 4921 | tgggtctcaa aaacactagg aacttttaag tgtcttagca cttcctcgat gtgcctgccc |
| 4981 | tgagggagtg agttcacatt tgagacaact gcactccagt gtggacgtgc ctttgtcttc |
| 5041 | aggccatgcc gaagggtgtt taaagcagtc ttgcaggtcg ctcctttccc agccgtggat |
| 5101 | aaaaactgaa gctaggaatc taataaggaa tgctgatttc ctcagttcca ttttgaggaa |
| 5161 | tggggaaggc tattctaaag aaaaaaatgg gatttgtttt ctcggcagat ctgcaaggct |
| 5221 | ggctttaaga gcacaaggag ggaaagtaac gaaagggctg gactactata aaagttacaa |
| 5281 | atacgtagtt agaccaatag atttatatag tcaggttttt gtcatgtaat ttattaacta |
| 5341 | actattacag aaacacagct aagaatatca agtatttctc tggctcttga cagaaaaaaa |
| 5401 | tcagttgact taaccctttg ctgtcaaaag agttggcgtt tcctgttctg ggtgctactg |
| 5461 | ccaaacgtta tggtacttag agtcgggatg cacaacttca accaccgact tatcaatgca |
| 5521 | gccgcctgtg tattgcaatt ggccgttacc ttaagcactg agccacccgg gtttagttca |
| 5581 | gccatttcaa gaagtatatt taacgtcggt agttctgctt tattaaaatg cagcagaggt |
| 5641 | actcttctgt cccttccgtt tatagttctc tgagagagtt ctattttttg gttttgtttt |
| 5701 | gtgttttctt ttgcattttg tatcttgtat ttatccctga acatgttttg tacctttttt |
| 5761 | tttttttttt ttaagaaaag gaattctttt gtgtatatat agatacttgc atgatatact |
| 5821 | gtagtcaatg ttcggttcct caaaaggtct tgctgctgtc aggtgttatg cactccatcc |
| 5881 | atcataactg tatgaaacac atttcatatg taaataaacg tgggacattt ggcccttgtg |
| 5941 | cttctgtgag agaattattg atggtgggtc tctgacatct ttgtgaagtt tgggaagtaa |
| 6001 | ttaattgcag cgacaagcta cagggtgttg cagaattctt cccactcaga agaatggcat |
| 6061 | attcgttctc attagtaatc agctattttg tcactttctt gttgactcca tcagtacatg |
| 6121 | ggtacaatcc gagggtgtga atttcagctt gaaattccat tgctgttcct tgttttgttt |
| 6181 | gtattgctct aagttgtatt cataatagca ctttcatatg tttctgcatt tgaaccttgc |
| 6241 | aataagcctg tgtggtaggc cacataggtc cgaataacct agttttacag ttgagggagc |
| 6301 | tgagctcaga ttcagttctt tgccgaagcc ctcatagctg gtaagtggct ttgcatatta |
| 6361 | gaacccaaat attttgctct ctaaatctaa tgctcgctct atgtggttat gtacatattg |
| 6421 | acaaatattc atttattcaa caaataaaaa gtatgtacaa aacaaaaaaa aaaaaaaaaa |
| 6481 | aaaaaaaaaa aaa |

In one aspect, the present invention relates to an isolated polynucleotide coding for a ZNF532-NUT fusion protein, wherein the isolated polynucleotide has the nucleic acid sequence set forth in SEQ ID NO.: 3. The amino acid sequence of the ZNF532-NUT fusion protein is set forth in SEQ ID NO.: 4. Detection of ZNF532-NUT can be used as a research and clinical diagnostic test in cancer management.

SEQ ID NO.: 3 is shown as follows:

ATGACCATGGGGG
ATATGAAGACCCCAGACTTTGATGACCTCCTGGCAGCATTTGACATCCCAGATATGGTCGATCCTAAAGC
AGCTATTGAGTCTGGACACGATGACCATGAAAGCCACATGAAGCAGAATGCTCACGGAGAGGATGACTCC
CACGCACCATCATCTTCTGATGTGGGTGTCAGCGTTATCGTCAAGAATGTTCGGAACATTGACTCTTCCG
AGGGCGGGGAGAAAGACGGCCACAACCCCACTGGCAATGGCTTACATAATGGGTTTCTCACAGCATCCTC
CCTTGACAGTTACAGTAAAGATGGAGCAAAGTCCTTGAAAGGAGATGTGCCTGCCTCTGAGGTGACACTG
AAAGACTCGACATTCAGCCAGTTTAGCCCGATCTCCAGTGCTGAAGAGTTTGATGACGACGAGAAGATTG
AGGTGGATGACCCCCCTGACAAGGAGGACATGCGATCAAGCTTCAGGTCGAATGTGTTGACGGGGTCGGC
TCCCCAGCAGGACTACGATAAGCTGAAGGCACTCGGAGGGGAAAACTCCAGCAAAACTGGACTCTCTACG
TCAGGCAATGTGGAGAAAAACAAAGCTGTTAAGAGAGAAACAGAAGCCAGTTCTATAAACCTGAGTGTTT
ATGAACCTTTTAAAGTCAGAAAAGCAGAGGATAAATTGAAGGAAAGCTCTGACAAGGTGCTGGAAAACAG
AGTCCTAGATGGGAAGCTGAGCTCCGAGAAGAATGACACCAGCCTCCCCAGCGTTGCGCCATCAAAGACA
AAGTCGTCCTCCAAGCTCTCGTCCTGCATCGCTGCCATCGCGGCTCTCAGCGCTAAAAAGGCGGCTTCAG
ACTCCTGCAAAGAACCAGTGGCCAATTCGAGGGAATCCTCCCCGTTACCAAAAGAAGTAAATGACAGTCC
GAGAGCCGCTGACAAGTCTCCTGAATCCCAGAATCTCATCGACGGGACCAAAAAACCATCCCTGAAGCAA
CCGGATAGTCCCAGAAGCATCTCAAGTGAGAACAGCAGCAAAGGATCCCCGTCCTCTCCCGCAGGGTCCA
CACCAGCAATCCCCAAAGTCCGCATAAAAACCATTAAGACATCTTCTGGGGAAATCAAGAGAACAGTGAC
CAGGGTATTGCCAGAAGTGGATCTTGACTCTGGAAAGAAACCTTCCGAGCAGACAGCGTCCGTGATGGCC
TCTGTGACATCCCTTCTGTCGTCTCCAGCATCAGCCGCCGTCCTTTCCTCTCCCCCCAGGGCGCCTCTCC
AGTCTGCGGTCGTGACCAATGCAGTTTCCCCTGCAGAGCTCACCCCCAAACAGGTCACAATCAAGCCTGT
GGCTACTGCTTTCCTCCCAGTGTCTGCTGTGAAGACGGCAGGATCCCAAGTCATTAATTTGAAGCTCGCT
AACAACACCACGGTGAAAGCCACGGTCATATCTGCTGCCTCTGTCCAGAGTGCCAGCAGCGCCATCATTA
AAGCTGCCAACGCCATCCAGCAGCAAACTGTCGTGGTGCCGGCATCCAGCCTGGCCAATGCCAAACTCGT
GCCAAAGACTGTGCACCTTGCCAACCTTAACCTTTTGCCTCAGGGTGCCCAGGCCACCTCTGAACTCCGC
CAAGTGCTAACCAAACCTCAGCAACAAATAAAGCAGGCAATAATCAATGCAGCAGCCTCGCAACCCCCCA
AAAAGGTGTCTCGAGTCCAGGTGGTGTCGTCCTTGCAGAGTTCTGTGGTGGAAGCTTTCAACAAGGTGCT
GAGCAGTGTCAATCCAGTCCCTGTTTACATCCCAAACCTCAGTCCTCCCGCCAATGCAGGGATCACGTTA
CCGACGCGTGGGTACAAGTGCTTGGAGTGTGGGGACTCCTTTGCACTTGAAAAGAGTCTGACCCAGCACT
ACGACAGACGGAGCGTGCGCATCGAAGTAACGTGCAACCATTGTACAAAGAACCTCGTTTTTTACAACAA
ATGCAGCCTCCTTTCCCATGCCCGTGGGCATAAGGAGAAAGGGGTGGTAATGCAATGCTCCCACTTAATT
TTAAAGCCAGTCCCAGCAGATCAAATGATAGTTTCTCCGTCAAGCAATACTTCCACTTCAACTTCCACTC
TTCAGAGCCCTGTGGGAGCTGGCACACACACTGTCACAAAAATTCAGTCTGGCATAACTGGGACAGTCAT
ATCGGCTCCTTCAAGCACTCCCATCACCCCAGCCATGCCCCTAGATGAAGACCCCTCCAAACTGTGTAGA
CATAGTCTAAAATGTTTGGAGTGTAATGAAGTCTTCCAGGACGAGACATCACTGGCTACACATTTCCAGC
AGGCTGCAGATAC
CTGCTCATATCCTTTGAGACAAGCTCTCTGGGCTGACCTAAAGGTAATGTGAGGTGCTACACCCCGTCAA
AGCATCTGCA
TTGCCGGGACCGGATATGAGCATGAAACCTAGTGCCGCCCTGTCTCCATCCCCTGCACTTCCCTTTCTCC
CACCAACTTCTGACCCACCAGACCACCCACCCAGGGAGCCACCTCCACAGCCCATCATGCCTTCAGTATT
CTCTCCAGACAACCCTCTGATGCTCTCTGCTTTCCCCAGCTCACTGTTGGTGACAGGGGACGGGGGCCCT
TGCCTCAGTGGGGCTGGGGCTGGCAAGGTCATTGTCAAAGTCAAGACAGAAGGGGGGTCAGCTGAGCCCT
CTCAAACTCAGAACTTTATCCTTACTCAGACTGCCCTCAATTCGACTGCCCCGGGCACTCCCTGTGGAGG
CCTTGAGGGTCCTGCACCTCCATTTGTGACAGCATCTAATGTGAAGACCATTCTGCCCTCTAAGGCTGTT
GGTGTCAGCCAGGAGGGTCCTCCAGGCCTTCCGCCTCAGCCTCCACCACCAGTTGCTCAACTGGTCCCCA
TTGTGCCCCTGGAAAAAGCTTGGCCAGGGCCACATGGGACAACCGGGGAAGGAGGTCCTGTGGCCACTCT
ATCCAAGCCTTCCCTAGGTGACCGCTCCAAAATTICCAAGGACGITTATGAGAACTICCGTCAGTGGCAG
CGTTACAAAGCCTTGGCCCGGAGGCACCTATCCCAGAGTCCTGACACAGAAGCTCTTTCCTGTTTTCTTA
TCCCAGTGCTTCGTTCCCTGGCCCGGCTGAAGCCCACTATGACCCTGGAGGAGGGACTGCCATTGGCTGT
GCAGGAGTGGGAGCACACCAGCAACTTTGACCGGATGATCTTTTATGAGATGGCAGAAAGGTTCATGGAG
TTTGAGGCTGAGGAGATGCAGATTCAGAACACACAGCTGATGAATGGGTCTCAGGGCCTGTCTCCTGCAA
CCCCTTTGAAACTTGATCCTCTAGGGCCCCTGGCCTCTGAGGTTTGCCAGCAGCCAGTGTACATTCCGAA
GAAGGCAGCCTCCAAGACACGGGCCCCCCGCCGGCGTCAGCGTAAAGCCCAGAGACCTCCTGCTCCTGAG
GCACCCAAGGAGATCCCACCAGAAGCTGTGAAGGAGTATGTTGACATCATGGAATGGCTGGTGGGGACTC
ACTTGGCCACTGGGGAGTCAGATGGAAAACAAGAGGAAGAAGGGCAGCAGCAGGAGGAGGAAGGGATGTA
TCCAGATCCAGGTCTCCTGAGCTACATCAATGAGCTGTGTTCTCAGAAGGTCTTTGTCTCCAAGGTGGAG
GCTGTCATTCACCCTCAATTTCTGGCAGATCTGCTGTCCCCAGAAAAACAGAGAGATCCCTTGGCCTTAA
TTGAGGAGCTAGAGCAAGAAGAAGGACTCACTCTTGCCCAGCTGGTCCAGAAGCGACTCATGGCCTTGGA
AGAGGAGGAAGATGCAGAGGCGCCTCCAAGTTTCAGTGGCGCTCAGTTGGACTCAAGTCCTTCTGGTTCT
GTTGAGGATGAAGATGGGGATGGGCGGCTTCGGCCCTCACCTGGGCTTCAGGGGGCTGGGGGCGCCGCTT
GCCTTGGAAAGGTTTCTTCTTCAGGAAAACGGGCAAGAGAAGTGCATGGTGGGCAGGAGCAAGCCCTAGA
TAGCCCCAGAGGGATGCACAGGGATGGGAACACTCTGCCATCCCCCAGCAGCTGGGACCTGCAGCCAGAA
CTTGCAGCTCCACAGGGAACTCCGGGACCCTTGGGTGTGGAGAGGAGAGGGTCTGGGAAGGTTATAAACC
AGGTATCTCTACATCAGGATGGCCATCTAGGAGGCGCTGGGCCTCCTGGGCACTGCCTGGTGGCTGATAG
GACTTCAGAGGCTCTGCCCCTTTGTTGGCAGGGAGGCTTCCAGCCTGAGAGCACTCCCAGTTTGGATGCT
GGACTTGCAGAGCTGGCTCCTCTGCAAGGACAAGGGTTAGAAAAGCAAGTCCTGGGATTGCAGAAAGGAC
AACAAACAGGGGGTCGTGGAGTGCTTCCTCAAGGGAAGGAGCCTTTAGCAGTGCCCTGGGAAGGCTCTTC
AGGAGCCATGTGGGGAGATGACAGAGGTACCCCCATGGCTCAGAGTTATGATCAGAATCCTTCCCCTAGA
GCAGCTGGGGAGAGGGACGATGTCTGTCTCAGCCCAGGAGTTTGGCTGAGCAGTGAGATGGATGCTGTAG
GCTTGGAGCTGCCTGTACAAATAGAGGAGGTCATAGAGAGCTTCCAAGTTGAGAAGTGTGTAACTGAGTA
TCAGGAAGGCTGCCAGGGACTGGGCTCCAGGGGCAACATTTCCCTGGGTCCTGGAGAAACCCTAGTACCT
GGGGATACGGAGAGCAGTGTGATTCCCTGTGGAGGCACAGTTGCGGCAGCTGCCCTAGAAAAGAGAAACT
ATTGCAGCTTGCCAGGACCTTTGAGGGCCAACAGCCCACCCTTGAGGTCCAAAGAAAATCAAGAACAGAG
CTGTGAAACCGTAGGGCATCCCAGTGATCTGTGGGCAGAAGGTTGCTTCCCATTGCTAGAAAGTGGTGAT
TCCACACTGGGGTCTTCCAAAGAAACCCTTCCACCCACATGCCAAGGCAATCTCCTTATCATGGGGACTG
AGGATGCCTCCTCCTTGCCTGAAGCCAGTCAAGAGGCAGGGAGCAGAGGCAATTCCTTTTCTCCTCTGTT
GGAAACCATAGAACCTGTCAACATACTAGATGTTAAAGATGACTGTGGCCTCCAACTAAGGGTCAGCGAG
GACACCTGCCCACTGAATGTTCATTCTTATGACCCCCAAGGAGAAGGCAGGGTGGATCCTGATCTGTCCA
AGCCTAAAAACCTTGCTCCTTTACAAGAGAGTCAGGAGTCTTACACAACTGGGACTCCCAAAGCAACATC
TTCTCACCAGGGCCTTGGAAGCACTTTGCCTAGAAGGGGAACCAGGAATGCCATAGTTCCGAGAGAAACT
TCTGTTAGTAAAACACACAGGTCAGCAGACAGGGCCAAAGGAAAGGAGAAAAAGAAAAAGGAAGCAGAGG
AAGAGGATGAGGAACTCTCCAACTTTGCTTACCTCTTGGCCTCTAAACTTAGCCTCTCACCAAGGGAGCA
TCCCCTCAGTCCTCACCATGCCTCAGGAGGTCAGGGCAGCCAGAGAGCATCCCACCTGCTCCCTGCTGGA
GCAAAAGGCCCCAGCAAACTTCCATATCCTGTTGCCAAGTCTGGGAAGCGAGCTCTAGCTGGAGGTCCAG
CCCCTACTGAAAAGACACCCCACTCAGGAGCTCAACTTGGGGTCCCCAGGGAGAAACCCCTAGCTCTGGG
AGTAGTTCGACCCTCACAGCCTCGTAAAAGGCGGTGTGACAGTTTTGTCACGGGCAGAAGGAAGAAACGA
CGTCGTAGCCAGTAG
GGAGCAGCGGGACCATCTGACCCCACTTGCCAGTCCCTAAAGGTGGGTGCCCCAG
AGTAGATTCCACCCCTGCTGCCCACCAATGGAGAATCCCAATGTTGAATCTCATCCCAATGTTGTTTTGT
TGTTCTGCAAAAGTGGCAAGCATGGAGAGAGAGGTCAGACTGGCTAGGCTGCAGGGGGAATTACCTTTGG
AAGGAGCTATATAGTGAAAAAAAATAAAGTGTTTTGTTGGAAAATGCTC

SEQ ID NO.: 4 is shown as follows:

MTMGDMKTPDFDDLLAAFDIPDMVDPKAAIESGHDDHESHMKQNAHGEDDSHAPSSSDVG
VSVIVKNVRNIDSSEGGEKDGHNPTGNGLHNGFLTASSLDSYSKDGAKSLKGDVPASEVT
LKDSTFSQFSPISSAEEFDDDEKIEVDDPPDKEDMRSSFRSNVLTGSAPQQDYDKLKALG
GENSSKTGLSTSGNVEKNKAVKRETEASSINLSVYEPFKVRKAEDKLKESSDKVLENRVL
DGKLSSEKNDTSLPSVAPSKTKSSSKLSSCIAAIAALSAKKAASDSCKEPVANSRESSPL
PKEVNDSPRAADKSPESQNLIDGTKKPSLKQPDSPRSISSENSSKGSPSSPAGSTPAIPK
VRIKTIKTSSGEIKRTVTRVLPEVDLDSGKKPSEQTASVMASVTSLLSSPASAAVLSSPP
RAPLQSAVVTNAVSPAELTPKQVTIKPVATAFLPVSAVKTAGSQVINLKLANNTTVKATV
ISAASVQSASSAIIKAANAIQQQTVVVPASSLANAKLVPKTVHLANLNLLPQGAQATSEL
RQVLTKPQQQIKQAIINAAASQPPKKVSRVQVVSSLQSSVVEAFNKVLSSVNPVPVYIPN
LSPPANAGITLPTRGYKCLECGSDSFALEKSLTQHYDRRVRIEVTCNHCTKNLVFYNKCS
LLSHARGHKEKGVVMQCSHLILKPVPADQMIVSPSSNTSTSTSTLQSPVGAGTHTVTKIQ
SGITGTVISAPSSTPITPAMPLDEDPSKLCRHSLKCLECNEVFQDETSLATHFQQAADTC
SYPLRQALWADLKECEVLHPVKASALPGPDMSMKPSAALSPSPALPFLPPTSDPPDHPPR
EPPPQPIMPSVFSPDNPLMLSAFPSSLLVTGDGGPCLSGAGAGKVIVKVKTEGGSAEPSQ
TQNFILTQTALNSTAPGTPCGGLEGPAPPFVTASNVKTILPSKAVGVSQEGPPGLPPQPP
PPVAQLVPIVPLEKAWPGPHGTTGEGGPVATLSKPSLGDRSKISKDVYENFRQWQRYKAL
ARRHLSQSPDTEALSCFLIPVLRSLARLKPTMTLEEGLPLAVQEWEHTSNFDRMIFYEMA
ERFMEFEAEEMQIQNTQLMNGSQGLSPATPLKLDPLGPLASEVCQQPVYIPKKAASKTRA
PRRRQRKAQRPPAPEAPKEIPPEAVKEYVDIMEWLVGTHLATGESDGKQEEEGQQQEEEG
MYPDPGLLSYINELCSQKVFVSKVEAVIHPQFLADLLSPEKQRDPLALIEELEQEEGLTL
AQLVQKRLMALEEEEDAEAPPSFSGAQLDSSPSGSVEDEDGDGRLRPSPGLQGAGGAACL
GKVSSSGKRAREVHGGQEQALDSPRGMHRDGNTLPSPSSWDLQPELAAPQGTPGPLGVER
RGSGKVINQVSLHQDGHLGGAGPPGHCLVADRTSEALPLCWQGGFQPESTPSLDAGLAEL
APLQGQGLEKQVLGLQKGQQTGGRGVLPQGKEPLAVPWEGSSGAMWGDDRGTPMAQSYDQ
NPSPRAAGERDDVCLSPGVWLSSEMDAVGLELPVQIEEVIESFQVEKCVTEYQEGCQGLG
SRGNISLGPGETLVPGDTESSVIPCGGTVAAAALEKRNYCSLPGPLRANSPPLRSKENQE
QSCETVGHPSDLWAEGCFPLLESGDSTLGSSKETLPPTCQGNLLIMGTEDASSLPEASQE
AGSRGNSFSPLLETIEPVNILDVKDDCGLQLRVSEDTCPLNVHSYDPQGEGRVDPDLSKP
KNLAPLQESQESYTTGTPKATSSHQGLGSTLPRRGTRNAIVPRETSVSKTHRSADRAKGK
EKKKKEAEEEDEELSNFAYLLASKLSLSPREHPLSPHHASGGQGSQRASHLLPAGAKGPS
KLPYPVAKSGKRALAGGPAPTEKTPHSGAQLGVPREKPLALGVVRPSQPRKRRCDSFVTG
RRKKRRRSQ

The polypeptide and coding nucleic acid sequences of ZNF532 of human origin and those of a number of animals are known in the art and are publically available, e.g., from the NCBI website.

In one aspect, the invention relates to a method of treating cancer in a subject comprising administering a pharmaceutically-effective amount of a ZNF532 inhibitor to the subject. In some embodiments, the cancer expresses ZNF532 protein. The ZNF532 protein can interact with BRD4 and/or NUT. In some embodiments, the cancer comprises a ZNF532-NUT fusion gene. In some embodiments, the cancer is NMC. In some embodiments, the cancer is Ewing sarcoma. In some embodiments, the cancer is head and neck squamous cell carcinoma.

In some embodiments, ZNF532 inhibitors as disclosed herein can be used to inhibit the cellular ZNF532 activity. In some embodiments, ZNF532 inhibitors as disclosed herein can decrease expression (level) of ZNF532. In some embodiments, the ZNF532 inhibitors inhibit the interaction of ZNF532 with BET proteins, such as BRD4 and BRD3. In some embodiments, the ZNF532 inhibitors inhibit interaction of ZNF532 with NUT.

The ability of a compound to inhibit ZNF532 can be assessed by measuring a decrease in activity of ZNF532 as compared to the activity of ZNF532 in the absence of a ZNF532 inhibitor. In some embodiments, the ability of a compound to inhibit ZNF532 can be assessed by measuring a decrease in the biological activity (e.g., protein activity), e.g., ZNF532-dependent enzyme activity, or decrease in ZNF532 expression as compared to the level of ZNF532 activity and/or expression in the absence of ZNF532 inhibitors.

In some embodiments, a ZNF532 inhibitor is a protein inhibitor, and in some embodiments, the inhibitor is any agent which inhibits the function of ZNF532 or the expression of ZNF532 from its gene. In some embodiments, a ZNF532 inhibitor is a gene silencing agent.

In some embodiments, a ZNF532 inhibitor can have an IC50 of less than 50 μM, e.g., a ZNF532 inhibitor can have an IC50 of from about 50 μM to about 5 nM, or less than 5 nM. For example, in some embodiments, a ZNF532 inhibitor has an IC50 of from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

In some embodiments, the ZNF532 inhibitor is a small molecule. As used herein, the term “small molecule” refers to a natural or synthetic molecule having a molecular mass of less than about 5 kD, organic or inorganic compounds having a molecular mass of less than about 5 kD, less than about 2 kD, or less than about 1 kD.

In some embodiments, the ZNF532 inhibitor can be an anti-ZNF532 antibody molecule or an antigen-binding fragment thereof. Suitable antibodies include, but are not limited to, polyclonal, monoclonal, chimeric, humanized, recombinant, single chain, Fab, Fab′, Fsc, Rv, and F(ab′)2 fragments. In some embodiments, neutralizing antibodies can be used as inhibitors of ZNF532. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies. In general, an antibody molecule obtained from humans can be classified in one of the immunoglobulin classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies provide high binding avidity and unique specificity to a wide range of target antigens and haptens. Monoclonal antibodies useful in the practice of the methods disclosed herein include whole antibody and fragments thereof and are generated in accordance with conventional techniques, such as hybridoma synthesis, recombinant DNA techniques and protein synthesis.

The ZNF532 polypeptide, or a portion or fragment thereof, can serve as an antigen, and additionally can be used as an immunogen to generate antibodies that immunospecifically bind the antigen, using standard techniques for polyclonal and monoclonal antibody preparation. Preferably, the antigenic peptide comprises at least 10 amino acid residues, or at least 15 amino acid residues, or at least 20 amino acid residues, or at least 30 amino acid residues. In some embodiments, the portion of the ZNF532 polypeptide used as an epitope is PAGSTPAIPKVRIKTIKTSSGEIKRTVTRVLPEVDLDSGKKPSEQTASVM (SEQ ID NO: 5).

Useful monoclonal antibodies and fragments can be derived from any species (including humans) or can be formed as chimeric proteins which employ sequences from more than one species. Human monoclonal antibodies or “humanized” murine antibody can also be used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction in the possibility of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is one option for minimizing the size of an antibody agent. ScFvs provide additional options for preparing and screening a large number of different antibody fragments to identify those that specifically bind. Techniques have been developed to display scFv molecules on the surface of filamentous phage that contain the gene for the scFv. scFv molecules with a broad range orantigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

Anti-ZNF532 antibodies are commercially available through vendors such as Santa Cruz Biotechnology, Sigma Aldrich, Abcam, Novus Biologicals, and Bethyl Laboratories.

In some embodiments, the ZNF532 inhibitor is a nucleic acid or a nucleic acid analog or derivative thereof, also referred to as a nucleic acid agent herein. As will be appreciated by those skilled in the art, the depiction of a single strand also defines the sequence of the complementary strand. Thus, a nucleic acid also encompasses the complementary strand of a depicted single strand.

Without limitation, the nucleic acid agent can be single-stranded or double-stranded. A single-stranded nucleic acid agent can have double-stranded regions, e.g., where there is internal self-complementarity, and a double-stranded nucleic acid agent can have single-stranded regions. The nucleic acid can be of any desired length. In particular embodiments, nucleic acid can range from about 10 to 100 nucleotides in length. In various related embodiments, nucleic acid agents, single-stranded, double-stranded, and triple-stranded, can range in length from about 10 to about 50 nucleotides, from about 20 to about 50 nucleotides, from about 15 to about 30 nucleotides, from about 20 to about 30 nucleotides in length. In some embodiments, a nucleic acid agent is from about 9 to about 39 nucleotides in length. In some other embodiments, a nucleic acid agent is at least 30 nucleotides in length.

The nucleic acid agent can comprise modified nucleosides as known in the art. Modifications can alter, for example, the stability, solubility, or interaction of the nucleic acid agent with cellular or extracellular components that modify activity. In certain instances, it can be desirable to modify one or both strands of a double-stranded nucleic acid agent. In some cases, the two strands will include different modifications. In other instances, multiple different modifications can be included on each of the strands. The various modifications on a given strand can differ from each other, and can also differ from the various modifications on other strands. For example, one strand can have a modification, and a different strand can have a different modification. In other cases, one strand can have two or more different modifications, and the another strand can include a modification that differs from the at least two modifications on the first strand.

Single-stranded and double-stranded nucleic acid agents that are effective in inducing RNA interference are referred to as siRNA, RNAi agents, iRNA agents, or RNAi inhibitors herein. As used herein, the term “iRNA agent” refers to a nucleic acid agent which can mediate the targeted cleavage of an RNA transcript via an RNA-induced silencing complex (RISC) pathway. ZNF532 RNAi agents are commercially available through vendors such as Novus Biologicals (Littleton, Colo.).

In some embodiments, the ZNF532 inhibitor is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

A small hairpin RNA or short hairpin RNA (shRNA) is a sequence of RNA that makes a tight hairpin turn that can be used to silence target gene expression via RNA interference (RNAi).

In general, any method of delivering a nucleic acid molecule can be adapted for use with the nucleic acid agents described herein.

Methods of delivering RNA interference agents, e.g., an siRNA, or vectors containing an RNA interference agent, to the target cells, for uptake include injection of a composition containing the RNA interference agent, e.g., an siRNA, or directly contacting the cell with a composition comprising an RNA interference agent, e.g., an siRNA. In another embodiment, RNA interference agent, e.g., an siRNA may be injected directly into any blood vessel, such as vein, artery, venule or arteriole, via, e.g., hydrodynamic injection or catheterization. Administration may be by a single injection or by two or more injections. The RNA interference agent is delivered in a pharmaceutically acceptable carrier. One or more RNA interference agents may be used simultaneously. In one embodiment, specific cells are targeted with RNA interference, limiting potential side effects. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, inhibit annealing of single strands, stabilize single strands, or otherwise facilitate delivery to the target cell and increase inhibition of the target gene, e.g., ZNF532 or ZNF532-NUT. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

In some embodiments, the ZNF532 inhibitor is a ZNF532 decoy molecule, or a polypeptide that blocks the binding of ZNF532 with NUT and/or BRD4.

In some embodiments, a BET inhibitor is administered to the subject in combination with the ZNF532 inhibitor to treat cancer. BET inhibitors are well known in the art and include, for example, but are not limited to JQ1, disclosed in WO/2009/084693 and GSK-525762A (also known as I-BET762, Wynce et al., Oncotarget. 2013; 4(12): 2419-2429. Inhibition of BET bromodomain proteins as a therapeutic approach in prostate cancer), LY294002 (Dittmann et al., “The Commonly Used PI3-Kinase Probe LY294002 is an Inhibitor of BET Bromodomains”. ACS Chemical Biology: 2013, 131210150813004.), OTX015 (Stathis A, et al., Cancer Discovery. 2016 in press), CPI-0610, and Volasertib (BI6727). BET inhibitors are also disclosed in US Application 2012/0208800 and International Applications WO201105484 and WO2006/032470 (SmithKline Beecham Corporation), which are each incorporated herein in their entirety. Such compounds can be prepared by methods described therein.

JQ1, also known as: (S)-tert-butyl 2-(4-(4-chlorophenyl)-2,3,9-trimethyl-6H-thieno[3,2-f][1,2,4]triazolo[4,3-a][1,4]diazepin-6-yl)acetate, and disclosed in WO/2009/084693, has the following structure:

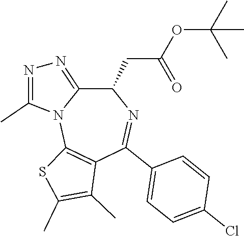 [SMILES: Cc1sc2c(c1C)C(c1ccc(Cl)cc1)=N[C@@H](CC(=O)OC(C)(C)C)c1nnc(C)n1-2]

I-BET-762 (also known as: GSK-525762A) is an inhibitor for BET proteins with IC50 of −35 nM, and suppresses the production of proinflammatory proteins by macrophages and blocks acute inflammation, highly selective over other bromodomain-containing proteins. I-BET-762 has the following structure:

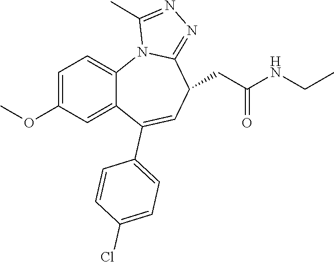 [SMILES: CCNC(=O)C[C@@H]1C=C(c2ccc(Cl)cc2)c2cc(OC)ccc2-n2c(C)nnc21]

LY294002 is the first synthetic molecule known to inhibit PI3Kα/δ/β with IC50 of 0.5 μM/0.57 μM/0.97 μM, respectively; and has been reported to be a BET inhibitor (Dittmann et al., “The Commonly Used PI3-Kinase Probe LY294002 is an Inhibitor of BET Bromodomains”. ACS Chemical Biology: 2013, 131210150813004). LY294002 has the following structure:

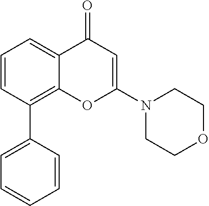 [SMILES: O=c1cc(N2CCOCC2)oc2c(-c3ccccc3)cccc12]

In a further embodiment a BET inhibitor is a compound that is generically or specifically disclosed in PCT publication WO2009/084693 (Mitsubishi Tanabe). Such compounds can be prepared by methods described therein. In a further embodiment the BET inhibitor is 1-[2-(1/-/-benzimidazol-2-ylthio)ethyl]-1,3-dihydro-3-methyl-2H-benzinidazole-2-thione as described in Japanese patent application JP2008-156311, which is incorporated herein in its entirety. It will be appreciated that a BET inhibitor used in the present invention may be in the form of a pharmaceutically acceptable salt, solvate (e.g. a hydrate) or prodrug or any other derivative of such a compound which upon administration to the recipient is capable of providing (directly or indirectly) the BET inhibitor of the invention, or an active metabolite or residue thereof. Suitable pharmaceutically acceptable salts can include acid or base addition salts. For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent. Suitable prodrugs are recognizable to those skilled in the art, without undue experimentation.

In one embodiment, the BET inhibitor is a small molecule. In one embodiment the BET inhibitor is a compound selected from the group consisting of the compounds shown in Table 1.

TABLE 1
Additional Exemplary BET inhibitors

| BET inhibitor | Structure |
|---|---|
| 1-methylethyl ((2S,4R)-1-acetyl-2-methyl-6-{4- [(methylamino)methyl]phenyl}-1,2,3,4-tetrahydro- 4-quinolinyl)carbamate | 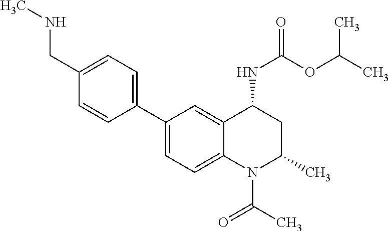 [SMILES: CNCc1ccc(-c2ccc3c(c2)[C@H](NC(=O)OC(C)C)C[C@H](C)N3C(C)=O)cc1] |
| 2-[(4S)-6-(4-Chlorophenyl)-1-methyl-8- (methyloxy)-4H-[1,2,4]triazolo[4,3- a][1,4]benzodiazepin-4-yl]-N-ethylacetamide | 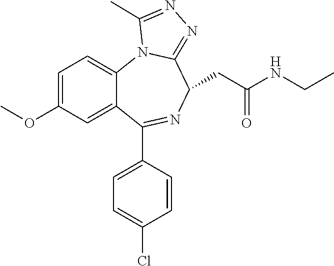 [SMILES: CCNC(=O)C[C@@H]1N=C(c2ccc(Cl)cc2)c2cc(OC)ccc2-n2c(C)nnc21] |
| 7-(3,5-dimethyl-4-isoxazolyl)-8-(methoxy)-1- [(1R)-1-(2-pyridinyl)ethyl]-1,3-dihydro-2H- imidazo[4,5-c]quinolin-2-one | 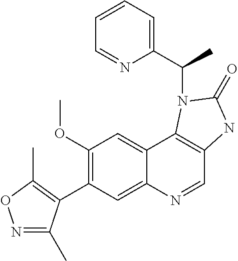 [SMILES: COc1cc2c(cc1-c1c(C)noc1C)ncc1[nH]c(=O)n([C@H](C)c3ccccn3)c12] |
| 7-(3,5-dimethyl-4-isoxazolyl)-8-(methoxy)-1- [(1R)-phenylethyl]-2-(tetrahydro-2H-pyran-4-yl)- 1H-imidazo[4,5-c]quinoline | 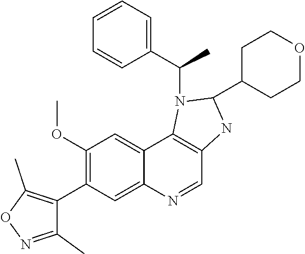 [SMILES: COc1cc2c3c(cnc2cc1-c1c(C)noc1C)NC(C1CCOCC1)N3[C@H](C)c1ccccc1] |
| 4-{(2S,4R)-1-acetyl-4-[(4-chlorophenyl)amino]-2- methyl-1,2,3,4-tetrahydro-6-quinolinyl}benzoic acid | 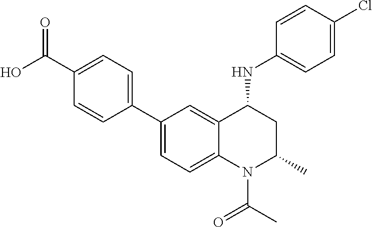 [SMILES: CC(=O)N1c2ccc(-c3ccc(C(=O)O)cc3)cc2[C@H](Nc2ccc(Cl)cc2)C[C@@H]1C] |
| N-{1-methyl-7-[4-(1- piperidinylmethyl)phenyl][1,2,4]triazolo[4,3- a]quinolin-4-yl}urea | 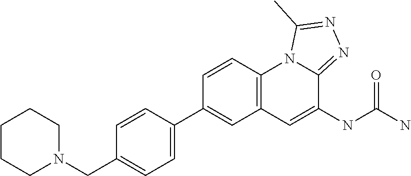 [SMILES: Cc1nnc2c(NC(N)=O)cc3cc(-c4ccc(CN5CCCCC5)cc4)ccc3n12] |

In some embodiments, the invention contemplates the practice of the method in conjunction with other therapies such as conventional chemotherapy directed against solid tumors and for control of establishment of metastases. The administration of the ZNF532 inhibitors described herein is typically conducted prior to and/or at the same time and/or after chemotherapy, although it is also encompassed within the present invention to inhibit cell proliferation after a regimen of chemotherapy at times where the tumor tissue will be responding to the toxic assault by inducing angiogenesis to recover by the provision of a blood supply and nutrients to the tumor tissue. In addition, the pharmaceutical compositions of the invention for the treatment of proliferative disorders, for example cancer, can be administrated prophylactically and/or before the development of a tumor, if the subject has been identified as to have a risk of developing cancer, for example to subjects that are positive for biomarkers of cancer cells or tumors. Insofar as the present methods apply to inhibition of cell proliferation, the methods can also apply to inhibition of tumor tissue growth, to inhibition of tumor metastases formation, and to regression of established tumors.

In some embodiments, the ZNF532 inhibitor described herein can be administered in the form of a pharmaceutical composition comprising the ZNF532 inhibitor, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a ZNF532 inhibitor as described herein.

The pharmaceutical compositions of the present invention can be specially formulated for administration in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally the compounds described herein can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments, the pharmaceutical composition comprising a ZNF532 inhibitor as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of a ZNF532 inhibitor as disclosed herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a ZNF532 inhibitor as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the ZNF532 inhibitor can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, a ZNF532 inhibitor as described herein can be administered in a liposome formulation. As used herein, “lipid vesicle” or “liposome” refers to vesicles surrounded by a bilayer formed of lipid components usually including lipids optionally in combination with non-lipidic components. The interior of a vesicle is generally aqueous. One major type of liposomal composition not generally found in nature includes phospholipids other than naturally-derived phosphatidylcholine. Neutral lipid vesicle compositions, for example, can be formed from dimyristoyl phosphatidylcholine (DMPC) or dipalmitoyl phosphatidylcholine (DPPC). Anionic lipid vesicle compositions generally are formed from dimyristoyl phosphatidylglycerol. Another type of liposomal composition is formed from phosphatidylcholine (PC) such as, for example, soybean PC, and egg PC. Another type is formed from mixtures of phospholipid and/or phosphatidylcholine and/or cholesterol. Lipids for lipid vesicle or liposome formation are known in the art or described herein below. Liposomes are formed by the self-assembly of phospholipid molecules in an aqueous environment. The amphipathic phospholipid molecules form a closed bilayer sphere in an attempt to shield their hydrophilic groups from the aqueous environment, while still maintaining contact with the aqueous phase via the hydrophilic head group. The resulting closed sphere can encapsulate aqueous soluble drugs or agents such as the hemoglobin, enzyme and cofactor compositions described herein, within the bilayer membrane. Non-limiting examples of liposome compositions include those described U.S. Pat. Nos. 4,983,397; 6,476,068; 5,834,012; 5,756,069; 6,387,397; 5,534,241; 4,789,633; 4,925,661; 6,153,596; 6,057,299; 5,648,478; 6,723,338; 6,627218; U.S. Pat. App. Publication Nos: 2003/0224037; 2004/0022842; 2001/0033860; 2003/0072794; 2003/0082228; 2003/0212031; 2003/0203865; 2004/0142025; 2004/0071768; International Patent Applications WO 00/74646; WO 96/13250; WO 98/33481; Papahadjopolulos D, Allen T M, Gbizon A, et al. “Sterically stabilized liposomes. Improvements in pharmacokinetics and antitumor therapeutic efficacy” Proc Natl Acad Sci U.S.A. (1991) 88: 11460-11464; Allen T M, Martin F J. “Advantages of liposomal delivery systems for anthracyclines” Semin Oncol (2004) 31: 5-15 (suppl 13). Weissig et al. Pharm. Res. (1998) 15: 1552-1556 each of which is incorporated herein by reference in its entirety.

In some embodiments, a ZNF532 inhibitor as described herein can be administered in an oral formulation. Pharmaceutical compositions comprising ZNF532 inhibitors of the present invention can also be formulated into oral dosage forms such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 22nd ed., Mack Publishing, Easton, Pa. (2012).

Typical oral dosage forms are prepared by combining the pharmaceutically acceptable salt of the disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents. Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent. Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-0-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The invention features an article of manufacture that contains packaging material and compounds of the present invention, for example a ZNF532 inhibitor as disclosed herein and/or functional derivatives thereof in a formulation contained within the packaging material. In some embodiments, a formulation can contain at least one of the compounds of the present invention, for example at least one ZNF532 inhibitor as disclosed herein and/or functional derivatives thereof and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The dosage can be determined by one of skill in the art and can also be adjusted by the individual physician in the event of any complication. Typically, the dosage of a composition comprising a ZNF532 inhibitor disclosed herein can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, or from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, or from 4.5 g/kg body weight to 5 g/kg body weight. Effective doses may be extrapolated from dose-response curves derived from in vitro or animal model test bioassays or systems. The dosage should not be so large as to cause unacceptable adverse side effects.

In certain embodiments, an effective dose of a composition comprising a ZNF532 inhibitor as described herein can be administered to a patient once. In certain embodiments, an effective dose of a composition comprising a ZNF532 inhibitor can be administered to a patient a few times or at a set schedule (e.g., once a day, twice a day, once every week, etc.).

In some embodiments, prior to the administration of the ZNF532 inhibitor, the method comprises requesting a test to measure a level of ZNF532 in a sample obtained from the subject. If the level of ZNF532 is found to be above a reference level, the ZNF532 inhibitor and/or BET inhibitor is administered to the subject. As used herein, the term “level of ZNF532” refers to a level of the ZNF532 protein, a level of a nucleic acid encoding the ZNF532 protein, a level of a fusion protein comprising ZNF532 (e.g., ZNF532-NUT), or a level of a nucleic acid encoding the fusion protein (e.g., ZNF532-NUT fusion gene). In some embodiments, the test further comprises determining the percentage of cells expressing ZNF532 in the sample. In some embodiments, when at least 10%, e.g., at least 20%, at least 30%, at least 40%, at least 50%, at least 60%, at least 70%, at least 80%, or at least 90% of the cells expressing ZNF532 above the reference level, the ZNF532 inhibitor and/or BET inhibitor is administered to the subject.

In some embodiments, the reference level can be the level of ZNF532 in a healthy subject or a population of healthy subjects. In some embodiments, the reference level can be the level of ZNF532 measured in a normal tissue.

In some embodiments, prior to the administration of the ZNF532 inhibitor, the method comprises requesting a test to determine whether a ZNF532-NUT fusion gene is present in a sample obtained from the subject. If the ZNF532-NUT fusion gene is found to be present in the sample, the ZNF532 inhibitor is administered to the subject.

Methods to measure gene expression products are well known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for ZNF532 are commercially available from vendors, and can be used for the purposes of the invention to measure protein expression levels. Alternatively, since the amino acid sequences for ZNF532 are known, one of skill in the art can raise their own antibodies against these polypeptides of interest for the purpose of the invention.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color or other readily detectable property, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA, “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique to detect the presence of an antibody or an antigen in a sample via enzymatic conversion of an indicator substrate generally to a visible or optically detectable form. The ELISA has been used as a diagnostic tool in medicine and plant pathology, as well as a quality control check in various industries.

In one embodiment, an ELISA involving at least one antibody with specificity for the particular desired antigen (e.g., ZNF532 as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

Commercial ELISA kits are available through vendors such as R&D Systems (McKinley Place, MN).

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of ZNF532 can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the ZNF532 gene. Such molecules can be isolated, derived, or amplified from a biological sample, such as a blood sample. Techniques for the detection of mRNA expression are known by persons skilled in the art, and can include, but are not limited to, PCR procedures, RT-PCR, quantitative RT-PCR Northern blot analysis, differential gene expression, RNA protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some related technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is labeled with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments, the ZNF532-NUT fusion gene can be measured by FISH. FISH uses fluorescent probes that bind to only those parts of the chromosome with which they show a high degree of sequence complementarity. In some embodiments, the ZNF532-NUT fusion gene can be measured by NanoString gene expression analysis. NanoString gene expression analysis is developed by NanoString Technology (Seattle, Wash., USA). NanoString is a multiplexed method for detecting gene expression and provides a method for direct measurement of mRNAs without the use of transcription or amplification. NanoString and aspects thereof are described in Geiss et al., “Direct multiplexed measurement of gene expression with color-coded probe pairs” Nature Biotechnology 26, 317-325 (2008); in U.S. Pat. Nos. 7,473,767, 7,941,279 and 7,919,237, and in U.S. Patent Application Publication No. 2010/0112710, the entire contents of each of which are hereby incorporated by reference. NanoString is also discussed in: Payton et al., “High throughput digital quantification of mRNA abundance in primary human acute myeloid leukemia samples” The Journal of Clinical Investigation 119(6): 1714-1726 (2009); and Vladislav et al. “Multiplexed measurements of gene signatures in different analytes using the NanoString nCounter Assay System” BMC Research Notes 2: 80 (2009), the entire contents of each of which are hereby incorporated by reference.

Without wishing to be bound by theory, because the oncogenic function of wild-type ZNF532 can be BRD4-dependent, ZNF532 can be a biomarker of tumor sensitivity to BET inhibitors. Accordingly, in yet another aspect, the invention relates to a method of determining whether a BET inhibitor therapy can be effective in a subject having cancer, the method comprising: measuring, in a sample obtained from the subject, a level of ZNF532; and determining that the therapy can be effective if the level of ZNF532 is above a reference level. In some embodiments, the method further comprises administering the BET inhibitor therapy to the subject when the level of ZNF532 is above the reference level.

In a related aspect, the invention relates to a method of determining whether a BET inhibitor therapy can be effective in a subject having cancer, the method comprising: determining, in a sample obtained from the subject, whether a ZNF532-NUT fusion gene is present in the sample; and determining that the therapy can be effective if the ZNF532-NUT fusion gene is present in the sample. In some embodiments, the method further comprises administering the BET inhibitor therapy to the subject when the ZNF532-NUT fusion gene is present in the sample.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are described herein.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “comprising” or “comprises” is used in reference to compositions, methods, and respective component(s) thereof, that are useful to an embodiment, yet open to the inclusion of unspecified elements, whether useful or not.

As used herein, the term “consisting essentially of” refers to those elements required for a given embodiment. The term permits the presence of elements that do not materially affect the basic and novel or functional characteristic(s) of that embodiment of the invention.

The term “NUT midline carcinoma” or “NMC” refers to a genetically defined, very aggressive epithelial cancer that usually arises in the midline of the body and is characterized by a chromosomal rearrangement in the nuclear protein in testis (NUT) gene. In approximately 75% of cases, the coding sequence of NUT on chromosome 15q14 is fused to BRD4 or BRD3, which creates a chimeric gene that encodes the BRD-NUT fusion protein. In the remaining cases, the fusion of NUT is to a partner gene, usually called NUT-variant which can include, but is no limited to, either BRD3, NSD3, or ZNF532. The symptoms of NMC are similar to other forms of cancer and dependent on the stage; while generalized symptoms (weight loss and fatigue) may be seen, site specific symptoms are also present. If the tumor involves the head and neck region (in about 35%), then pain, a mass, obstructive symptoms, among others, may be experienced. NUT midline carcinomas are not specific to any tissue type or organ; common sites include the head, neck and mediastinum. The median age at diagnosis is 17 years, but older patients may be affected.

The term “ZNF532 inhibitor” generally refers to an agent or molecule that inhibits the activity or expression of ZNF532 or a fusion protein comprising ZNF532 (e.g., ZNF532-NUT). ZNF532 inhibitors can be of synthetic or biological origins. They can be organic, or inorganic molecules, or peptides, antibodies or antisense RNA that inhibit ZNF532. Inhibitors of ZNF532 of the invention are chemical entities or molecules that can inhibit expression of ZNF532 or ZNF532-NUT, and/or biological activity of ZNF532 or ZNF532-NUT, and/or the interaction of ZNF532 with BET proteins such as BRD4 and/or BRD3. ZNF532 inhibitors include, for example, RNAi agents, antisense nucleic acids, dominant negative proteins (decoy molecules), large polypeptides, or modified RNA (modRNA) which express decoy proteins, and enantiomers, prodrugs, derivatives and pharmaceutically acceptable salts thereof, which are discussed further in the section.

As used herein, the term “BET inhibitor” or “BETi” denotes a compound which inhibits the binding of a bromodomain with its cognate acetylated proteins. In one embodiment, the BET inhibitor is a compound which inhibits the binding of a BET protein to acetylated lysine residues. In a further embodiment, the BET inhibitor is a compound which inhibits the binding of a BET protein to acetylated lysine residues on histones, particularly histones H3 and H4. In a particular embodiment, the BET inhibitor is a compound that inhibits the binding of BET family bromodomains to acetylated lysine residues (hereafter referred to as a “BET family bromodomain inhibitor”). In one embodiment, the BET family bromodomain is BRD2, BRD3 or BRD4. A BET family bromodomain inhibitor is a compound which has a plC50≥5.0 of at least in one or more of the binding assays, or described in International Patent Application WO2013/026874, which is incorporated herein in its entirety by reference.

As used herein, the terms “nucleic acid,” “polynucleotide,” and “oligonucleotide” generally refer to any polyribonucleotide or poly-deoxyribonucleotide, and includes unmodified RNA, unmodified DNA, modified RNA, and modified DNA. Polynucleotides include, without limitation, single- and double-stranded DNA and RNA polynucleotides. The term polynucleotide, as it is used herein, embraces chemically, enzymatically or metabolically modified forms of polynucleotides, as well as the naturally occurring chemical forms of DNA and RNA found in or characteristic of viruses and cells, including for example, simple (prokaryotic) and complex (eukaryotic) cells. A nucleic acid polynucleotide or oligonucleotide as described herein retains the ability to hybridize to its cognate complimentary strand.

As used herein, “gene silencing” or “gene silenced” in reference to an activity of an RNAi molecule, for example a siRNA or miRNA refers to a decrease in the mRNA level in a cell for a target gene (e.g. ZNF532 gene or ZNF532-NUT fusion gene) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to inhibit the ZNF532 gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “gene product(s)” as used herein refers to include RNA transcribed from a gene, or a polypeptide encoded by a gene or translated from RNA.

The terms “lower”, “reduced”, “reduction” or “decrease”, “down-regulate” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease” or “inhibition” is used in the context of the level of expression or activity of a gene or a protein, e.g. ZNF532, it refers to a reduction in protein or nucleic acid level or activity in a cell, a cell extract, or a cell supernatant. For example, such a decrease may be due to reduced RNA stability, transcription, or translation, increased protein degradation, or RNA interference. In some embodiments, a ZNF532 inhibitor which is a small molecule as disclosed herein can decrease the activity or expression of ZNF532. Preferably, this decrease is at least about 5%, at least about 10%, at least about 25%, at least about 50%, at least about 75%, at least about 80%, or even at least about 90% of the level of expression or activity under control conditions. The term “level” as used herein in reference to ZNF532 refers to expression or activity of ZNF532.

The terms “subject” and “individual” are used interchangeably herein, and refer to an animal, for example a human, to whom treatment for cancer or a proliferative disorder, including therapeutic treatment or prophylactic treatment, with a pharmaceutical composition comprising a ZNF532 inhibitor or BET inhibitor as disclosed herein can be administered. The term “subject” as used herein includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

As used herein, the term “cancer” refers to an uncontrolled growth of cells which interferes with the normal functioning of the bodily organs and systems. A subject who has a cancer is a subject having objectively measurable cancer cells present in the subject's body. Included in this definition are benign and malignant cancers, premalignant lesions, as well as dormant tumors or micrometastases. Cancers which migrate from their original location and seed vital organs can eventually lead to the death of the subject through the functional deterioration of the affected organs.

As used herein, the terms “treat” or “treatment” or “treating” refers to therapeutic treatment, wherein the object is to prevent or slow the development of the disease, such as slow down the development of a tumor, the spread of cancer, or reducing at least one effect or symptom of a condition, disease or disorder associated with inappropriate proliferation or a cell mass, for example cancer. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer, as well as those likely to develop secondary tumors due to metastasis.

The term “prophylactic treatment” refers to the prevention of the development of cancer in a subject when the subject is at a high risk of developing cancer, such as, for example, a predisposition to cancer where the subject has a genetic mutation or polymorphism known to increase occurrence of a cancer, or a family history of cancer. In some embodiments, prophylactic treatment is used in a subject who has been successfully therapeutically treated for cancer and where the cancer has been eliminated or the subject has gone into remission, and is administered prophylactic treatment with comprising a ZNF532 inhibitor or BET inhibitor to prevent a cancer relapse.

The term “pharmaceutically-effective amount” as used herein refers to the amount of a pharmaceutical composition comprising a ZNF532 inhibitor or BET inhibitor as disclosed herein, to alleviate at least one or more symptom of the disease or disorder, and relates to a sufficient amount of pharmacological composition to provide the desired effect, e.g., to stop or reduce or lessen at least one symptom of the disease or disorder (e.g., cancer). The term also means a sufficient amount of the composition to treat a disorder, at a reasonable benefit/risk ratio applicable to any medical treatment, or an amount of the composition as disclosed herein that is sufficient to effect a therapeutically or prophylactically significant reduction in a symptom or clinical marker associated with a cancer or a cancer-mediated condition.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 95% in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

As used herein, the term “pharmaceutical composition” refers to the active agent in combination with a pharmaceutically acceptable carrier e.g. a carrier commonly used in the pharmaceutical industry.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intrahepatic, intraperitoneal, transtracheal, subcutaneous, subcuticular, intraarticular, sub capsular, subarachnoid, intraspinal, intracerebro spinal, and intrasternal injection and infusion. The administration can be systemic or local.

The term “sample”, “biological sample”, or “test sample” as used herein denotes a sample taken or isolated from a biological organism, e.g., a cancer sample from a subject. Exemplary biological samples include, but are not limited to, a biofluid sample; serum; plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, the test sample can be a cancer biopsy.

The test sample can be obtained by removing a sample from a subject, but can also be accomplished by using previously isolated sample (e.g. isolated at a prior time point and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing the methods described herein. After thawing, a frozen sample can be centrifuged before being subjected to the methods described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages may mean±1% of the value being referred to. For example, about 100 means from 99 to 101.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the invention. Further, all patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Embodiments of various aspects described herein can be defined in any of the following numbered paragraphs:

1. A method of treating cancer in a subject comprising administering a pharmaceutically-effective amount of a ZNF532 inhibitor to the subject.
2. The method of paragraph 1, wherein the cancer expresses ZNF532 protein or comprises a ZNF532-NUT fusion gene.
3. The method of paragraph 2, wherein the cancer is selected from the group consisting of NUT midline carcinoma (NMC), Ewing sarcoma, and head and neck squamous cell carcinoma.
4. The method of any one of paragraphs 1-3, wherein the ZNF532 inhibitor is selected from the group consisting of a small molecule, an antibody, an antibody fragment, RNAi, siRNA, a ZNF532 decoy molecule, a polypeptide that blocks the binding of ZNF532 with NUT and/or BRD4.
5. The method of any one of paragraphs 1-4, wherein the subject is a mammal.
6. The method of paragraph 5, wherein the mammal is a human.
7. A method of treating cancer in a subject comprising requesting a test to measure a level of ZNF532 in a sample obtained from the subject, and administering to the subject a pharmaceutically-effective amount of a ZNF532 inhibitor and/or a BET inhibitor in response to the level of ZNF532 above a reference level.
8. A method of treating cancer in a subject comprising requesting a test to determine whether a ZNF532-NUT fusion gene is present in a sample obtained from the subject, and administering to the subject a pharmaceutically-effective amount of a ZNF532 inhibitor and/or a BET inhibitor in response to the ZNF532-NUT fusion gene identified to be present in the sample.
9. The method of paragraph 7 or 8, wherein the cancer is selected from the group consisting of NUT midline carcinoma (NMC), Ewing sarcoma, and head and neck squamous cell carcinoma.
10. The method of any one of paragraphs 7-9, wherein the ZNF532 inhibitor is selected from the group consisting of a small molecule, an antibody, an antibody fragment, RNAi, siRNA, a ZNF532 decoy molecule, a polypeptide that blocks the binding of ZNF532 with NUT and/or BRD4.
11. The method of any one of paragraphs 7-10, wherein the BET inhibitor is selected from the group consisting of: JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), GSK-525762A, LY294002, 1-[2-(1/-/-benzimidazol-2-ylthio)ethyl]-1,3-dihydro-3-methyl-2H-benzinidazole-2-thione, 1-methylethyl ((2S,4R)-1-acetyl-2-methyl-6-{4-[(methylamino)methyl]phenyl}-1,2,3,4-tetrahydro-4-quinolinyl)carbamate, 2-[(4S)-6-(4-Chlorophenyl)-1-methyl-8-(methyloxy)-4H-[1,2,4]triazolo [4,3-a][1,4]benzodiazepin-4-yl]-N-ethylacetamide, 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one, 7-(3,5-dimethyl-4-isoxazolyl)-8-(methoxy)-1-[(1R)-phenylethyl]-2-(tetrahydro-2H-pyran-4-yl)-1H-imidazo[4,5-c]quinolone, 4-{(2S,4R)-1-acetyl-4-[(4-chlorophenyl)amino]-2-methyl-1,2,3,4-tetrahydro-6-quinolinyl}benzoic acid, and N-{1-methyl-7-[4-(1-piperidinylmethyl)phenyl][1,2,4]triazolo [4,3-a]quinolin-4-yl}urea, OTX015, CPI-0610, and Volasertib.
12. The method of any one of paragraphs 7-11, wherein the sample is a cancer sample.
13. The method of any one of paragraphs 7-12, wherein the subject is a mammal.
14. The method of paragraph 13, wherein the mammal is a human.
15. Use of a ZNF532 inhibitor for the manufacture of a medicament for treating cancer, wherein the cancer expresses ZNF532 protein or comprises a ZNF532-NUT fusion gene.
16. Use of a BET inhibitor for the manufacture of a medicament for treating cancer, wherein the cancer expresses ZNF532 protein or comprises a ZNF532-NUT fusion gene.
17. A method of determining whether a BET inhibitor therapy can be effective in a subject having cancer, the method comprising:
(i) measuring, in a sample obtained from the subject, a level of ZNF532; and
(ii) determining that the therapy can be effective if the level of ZNF532 is above a reference level.
18. The method of paragraph 17, further comprising administering the BET inhibitor therapy to the subject when the level of ZNF532 is above the reference level.
19. A method of determining whether a BET inhibitor therapy can be effective in a subject having cancer, the method comprising:
(i) determining, in a sample obtained from the subject, whether a ZNF532-NUT fusion gene is present in the sample; and
(ii) determining that the therapy can be effective if the ZNF532-NUT fusion gene is present in the sample.
20. The method of paragraph 19, further comprising administering the BET inhibitor therapy to the subject when the ZNF532-NUT fusion gene is present in the sample.
21. The method of any one of paragraphs 17-20, wherein the sample is a cancer sample.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example 1

A novel zinc finger protein that interacts with BRD4-NUT on chromatin, ZNF532 was identified. Expression of ZNF532 protein appeared to be restricted to NMC, Ewing sarcoma, HNSQC, and was low or absent in normal tissues, implicating its potential as a specific diagnostic and oncogenic therapeutic target. Additionally, a novel ZNF532-NUT fusion was discovered in a patient with NMC, underscoring the functional importance of ZNF532 in NMC pathogenesis. It was demonstrated that ZNF532-NUT and wild type ZNF532 are required for the blockade of differentiation in ZNF532-NUT+ and BRD4-NUT+NMCs, respectively. ZNF532-NUT function was found to be dependent upon BRD4. Analogous to ZNF532-NUT, it was previously demonstrated that another novel fusion oncoprotein in NMC, NSD3-NUT, localizes to chromatin via interaction with endogenous BRD4 (4). ZNF532-NUT may also target NUT to chromatin via interaction with BRD4.

Without wishing to be bound by theory, the ZNF532 portion of ZNF532-NUT is important to the formation of BRD4-NUT-like complexes by interaction with endogenous BRD4; and wild type ZNF532 may determine the DNA-binding specificity of BRD4 and BRD4-NUT.

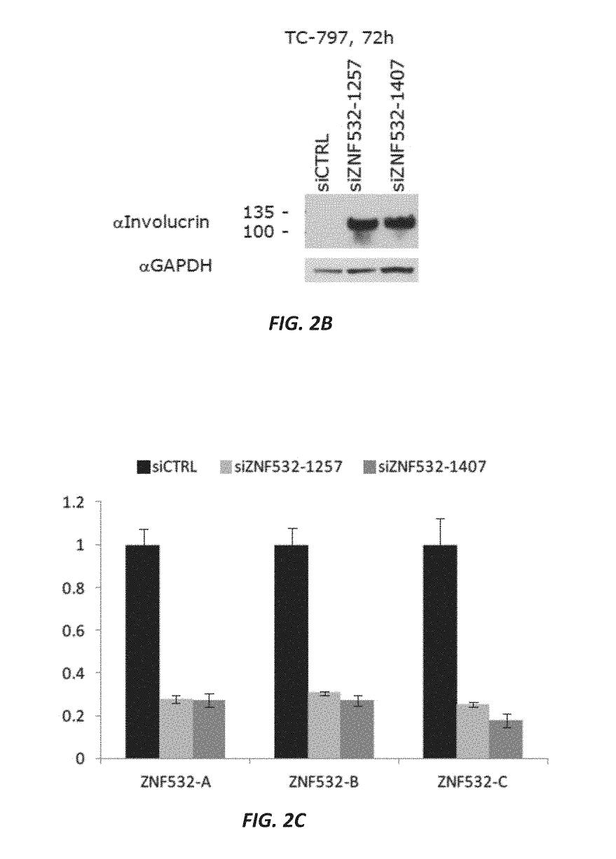

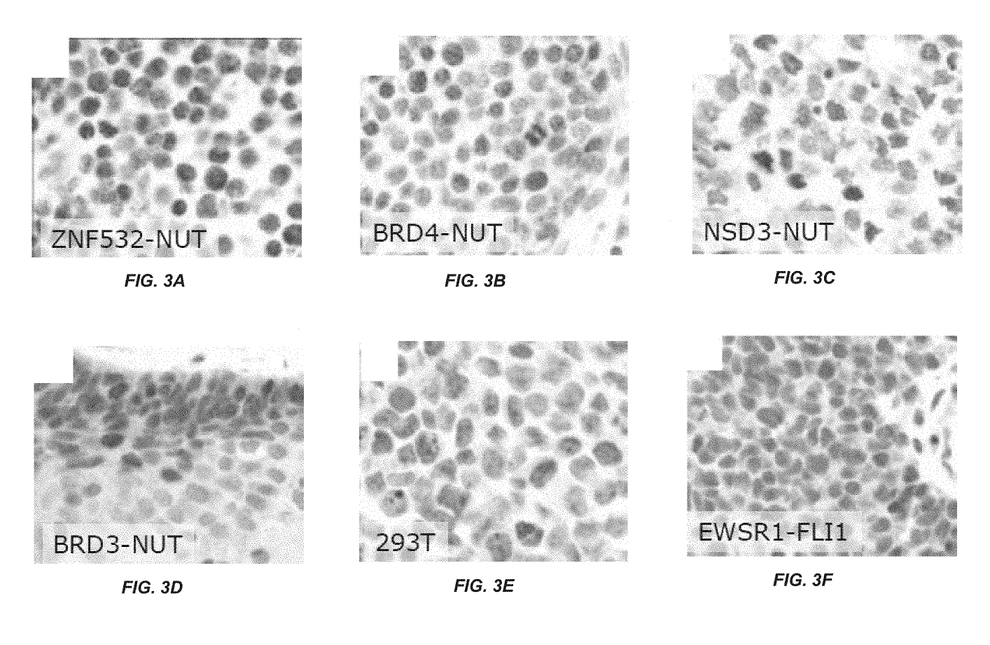

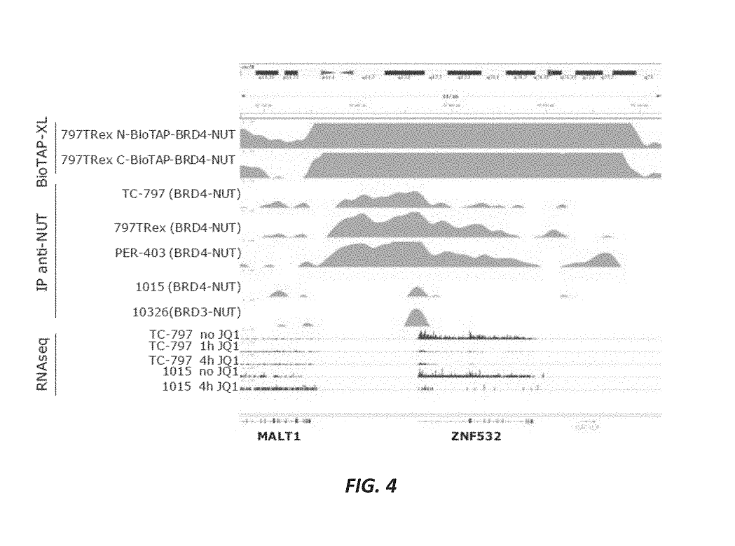

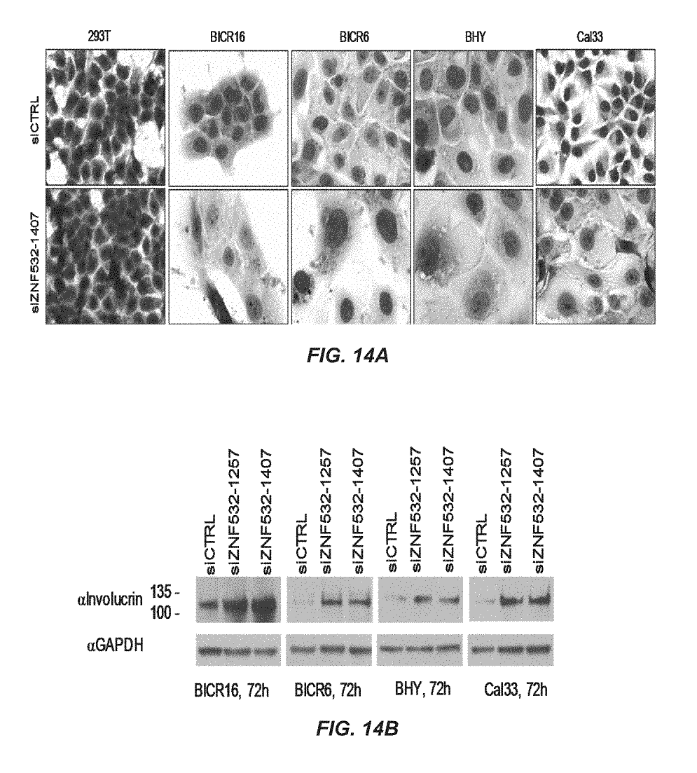

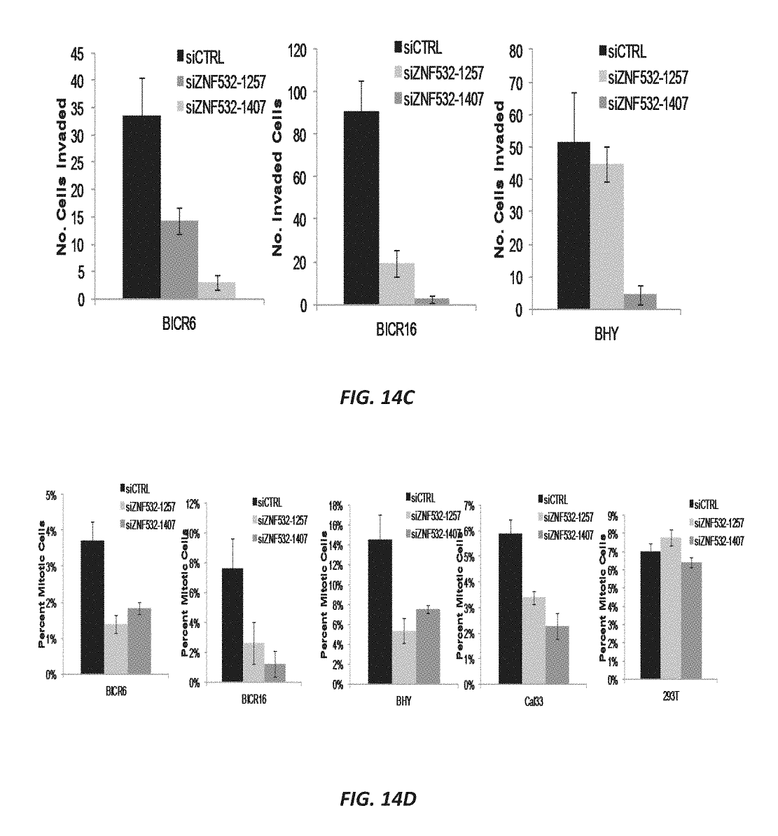

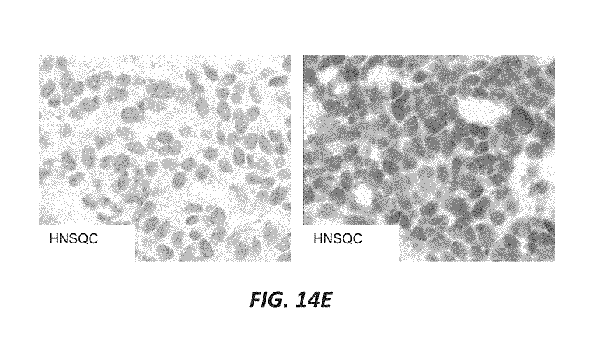

A strategy termed BioTAP-XL (7), was used to identify components of chromatin-associated BRD4-NUT protein complexes by affinity-purification and mass-spectrometry, and ChIP-seq of crosslinked chromatin. It was discovered that BRD4-NUT associates with at least 28 transcriptional activator proteins, including all and more than those which have taken over a decade to identify (BRD3, BRD2, CDK9, GLTSCR1, JMJD6, NSD1, NSD2, NSD3, ATAD5, MED1, MED24, MED23(8, 9), and p300(10)), and establishes unprecedented, 100 kb-2 MB ‘megadomains’ of active chromatin (Table 2). BRD4-NUT, histone H3 acetyl-lysine 27, and p300 co-occupy these megadomains, which activate transcription of underlying coding and non-coding DNA to regulate the expression of neighboring key oncogenic drivers of NMC. MYC is one critical oncogenic target in NMC (11) whose expression is consistently maintained by a megadomain through the BET-inhibitor-sensitive transcription of flanking long non-coding RNAs, CCAT1 and PVT1. Amongst the previously unreported proteins that were associated with BRD4-NUT is a zinc finger protein (ZNF532) of unknown function. Consistent with its association with BRD4-NUT, ZNF532 co-localizes with HA-tagged BRD4-NUT by immunofluorescence (FIG. 2A). siRNA knockdown of ZNF532 in the BRD4-NUT+cell line, TC-797(12), results in rapid terminal differentiation and arrested proliferation, indicating that ZNF532 is required for the blockade of differentiation in NMC (FIG. 2B). ZNF532 protein was expressed in all NMCs tested (N=10) and appears to be autoregulated by BRD4-NUT in a BET-inhibitor-sensitive manner (FIGS. 3A-3F & 4). Conversely, the expression of ZNF532 was found to be very low or absent in normal tissues, based on immunohistochemistry in 22 different tissues and MOPED, PaxDb, and MaxQB protein expression data. In contrast, ZNF532 was robustly expressed in the one Ewing sarcoma tested (FIG. 3F), and 3 of 12 (25%) of HNSQCs (FIG. 14E).

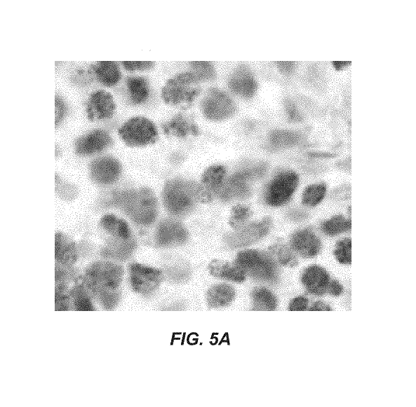

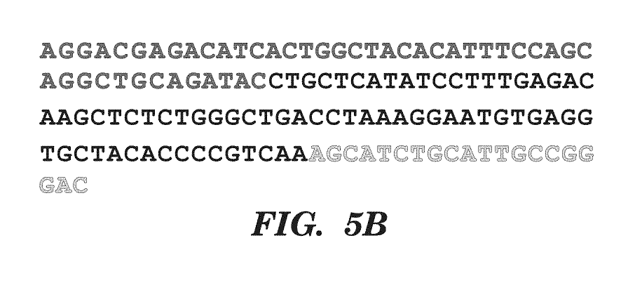

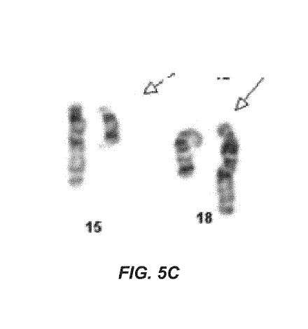

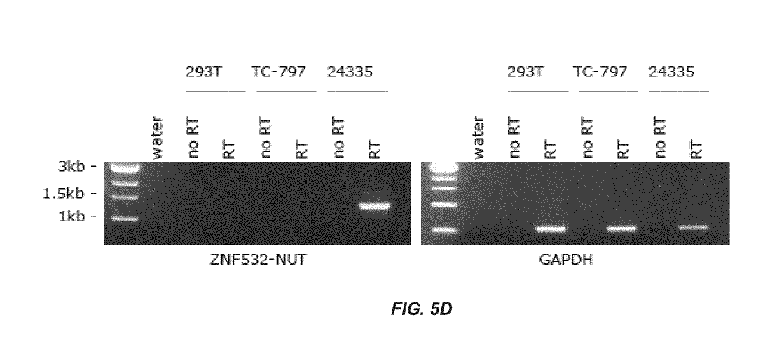

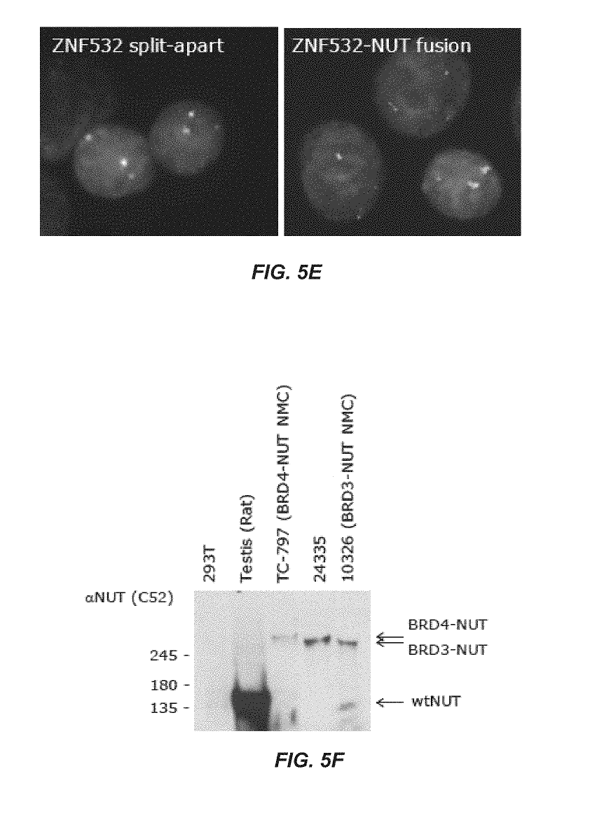

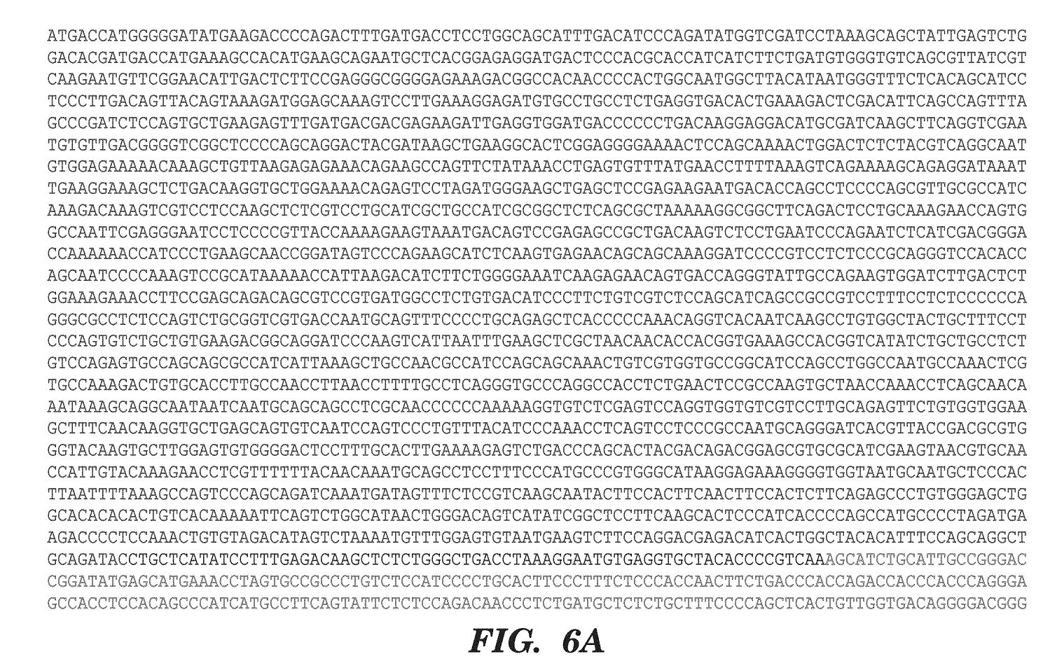

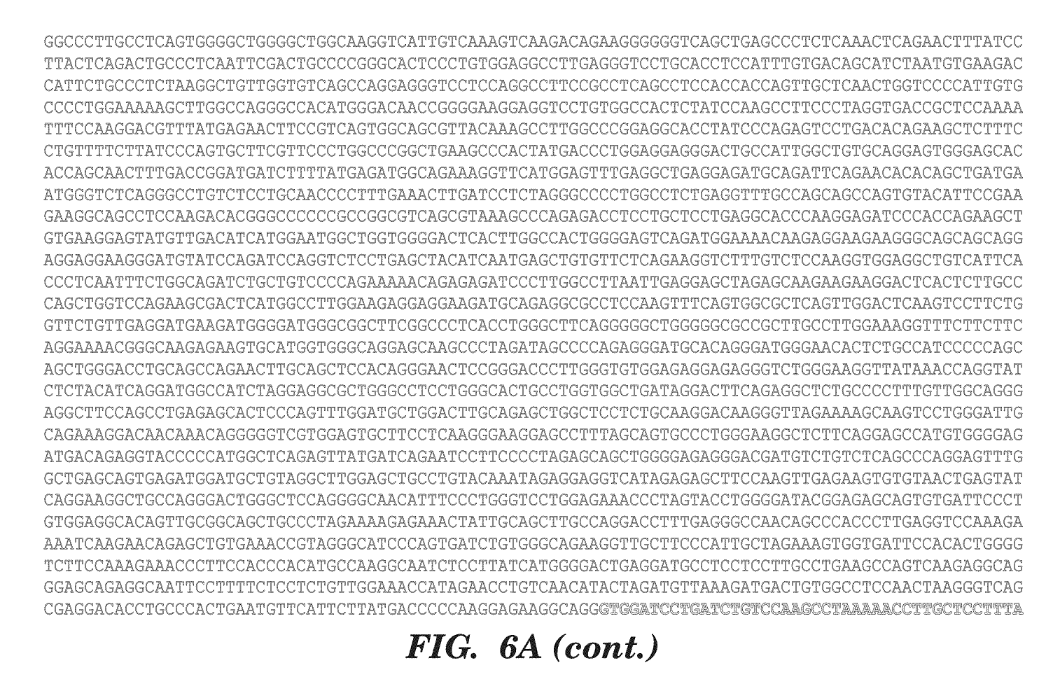

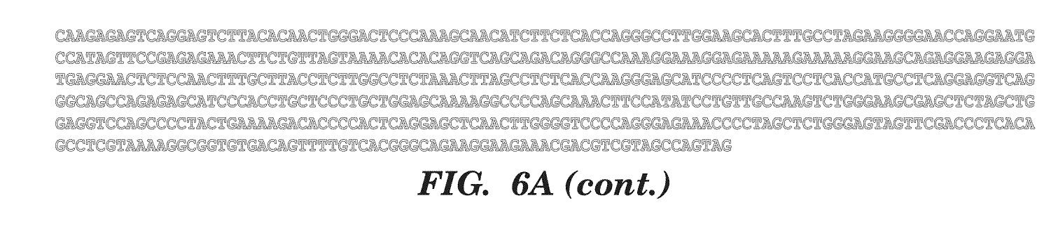

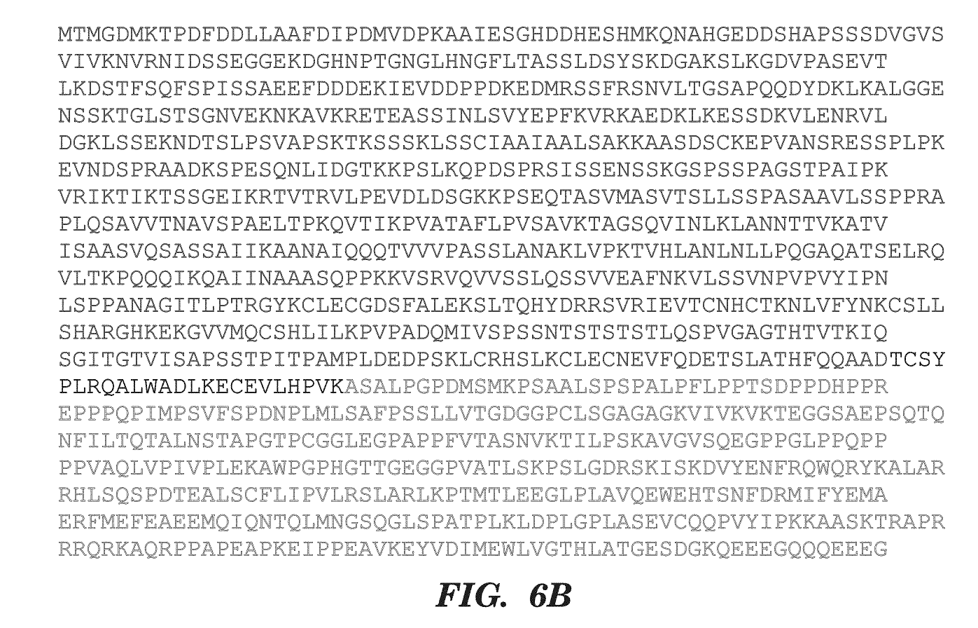

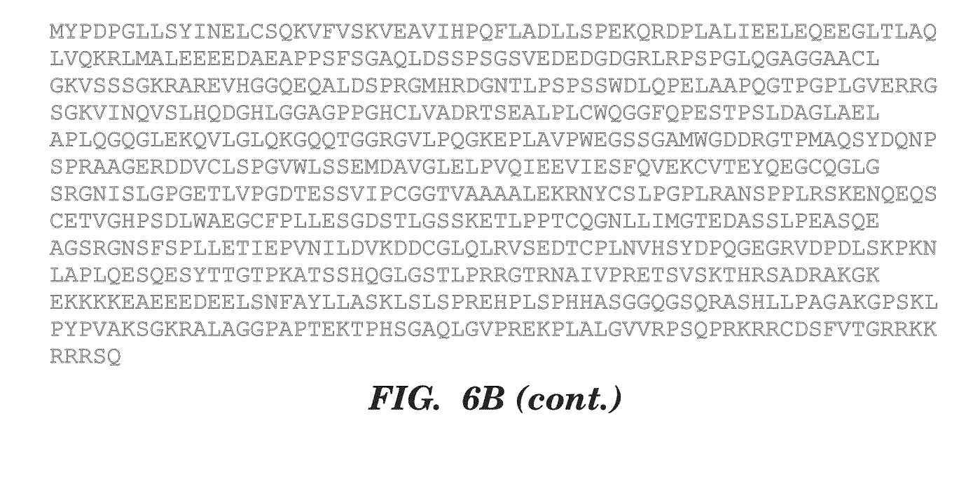

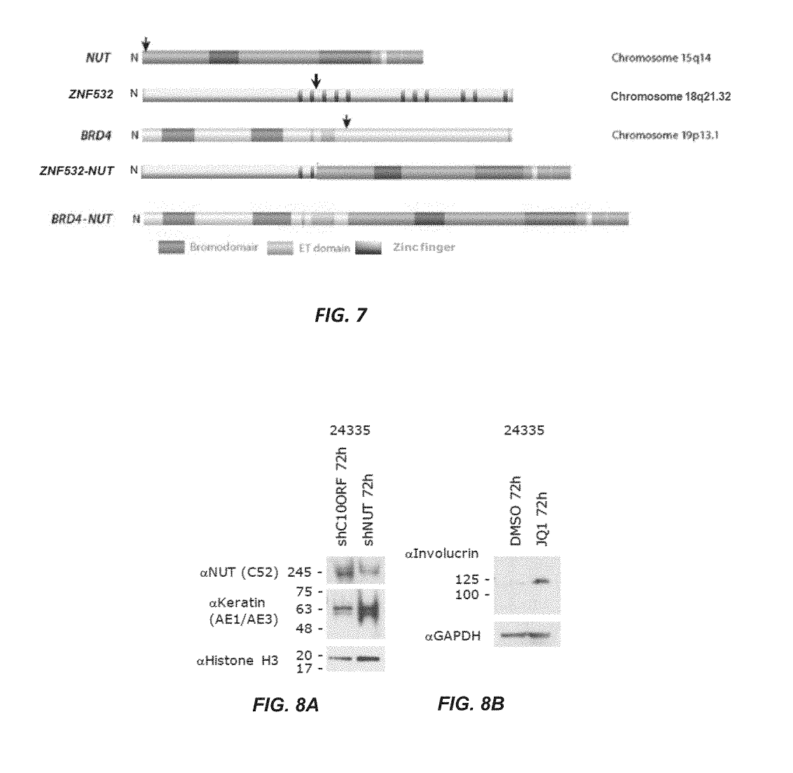

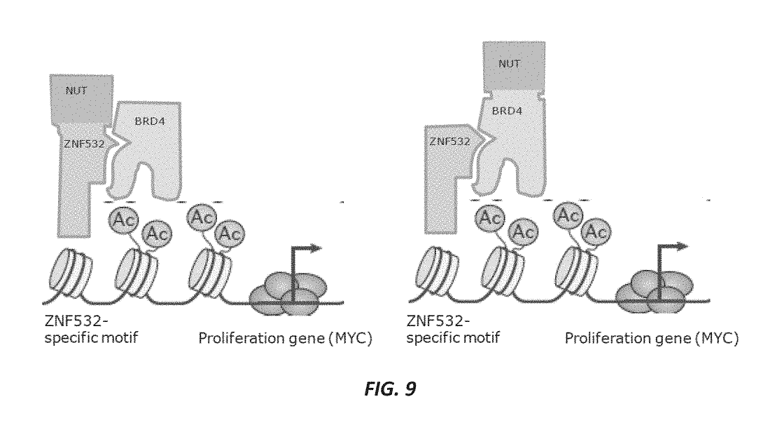

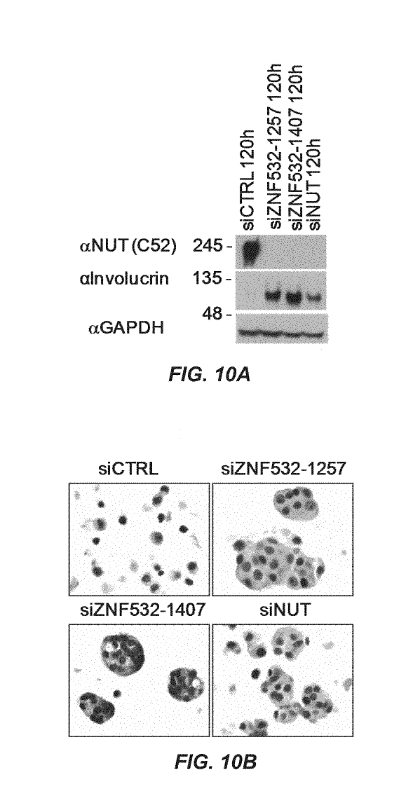

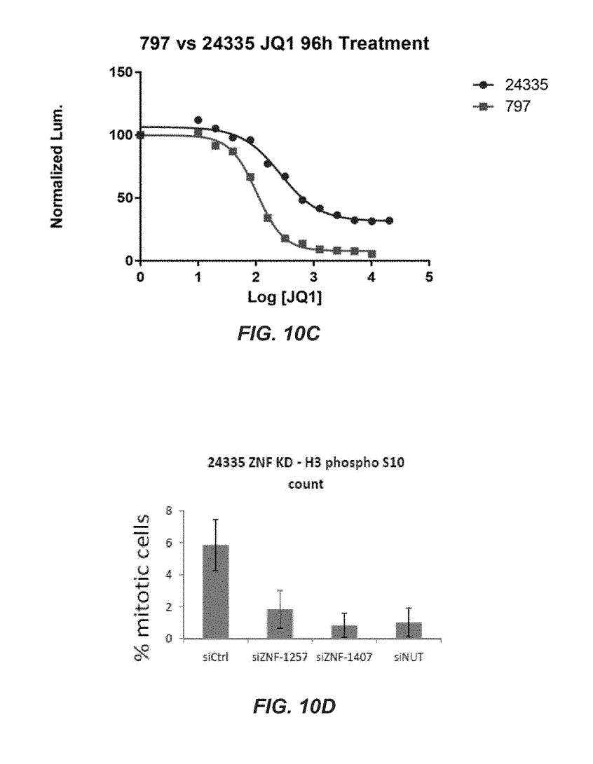

The importance of ZNF532 in NMC was underscored by simultaneous discovery of a 61 year old female patient with pulmonary NMC (FIG. 5A) harboring a ZNF532-NUT fusion gene, that was identified by whole transcriptome RNAsequencing (FIG. 5B), and confirmed by cytogenetics, fluorescence in situ hybridization (FISH), RT-PCR, western blotting, and immunofluorescence (FIGS. 5C-5F, 6). The fusion gene is comprised of exon 3 of ZNF532 fused to intron 1 of NUT (FIG. 7). The resulting predicted 1500 amino acid protein includes the N-terminal 778 amino acids of ZNF532, encoding only the first two of eleven zinc fingers and a large unstructured domain, part of intron 1 of NUT, and its remaining exons 2-7 (FIGS. 8A-8B). The first zinc finger included in the ZNF532-NUT fusion encodes a putative zinc ribbon domain that is predicted to bind nucleic acids directly. Exons 2-7 of NUT are recurrently included in all previously documented NUT-fusions. In an analysis of the cell line (24335) derived from this patient, we found that either ZNF532-NUT knockdown or treatment with BET-inhibitors results in differentiation and arrested proliferation of 24335 (FIGS. 8A-8B, 10A-C). These findings indicate that ZNF532-NUT is required for the blockade of differentiation and maintenance of proliferation of ZNF532-NUT+NMCs, in a BRD4-dependent manner. Thus, ZNF532-NUT+NMCs can be treated with BET inhibitors. The oncogenic function of wild-type ZNF532 can be BRD4-dependent, and thus ZNF532 can be a biomarker of tumor sensitivity to BET inhibitors.

ZNF532-NUT is a novel fusion oncogene in NMC. We have identified a means to diagnose ZNF532-NUT fusions, either by FISH or nanostring, ZNF532 is required for the blockade of differentiation and maintenance of proliferation in NMC (both BRD4-NUT+ and ZNF532-NUT+). ZNF532 can be a therapeutic target in NMC (both BRD4-NUT+ and ZNF532-NUT+) and HNSQC . . . ZNF532 is a biomarker for response of any cancer to BET inhibitor therapy.

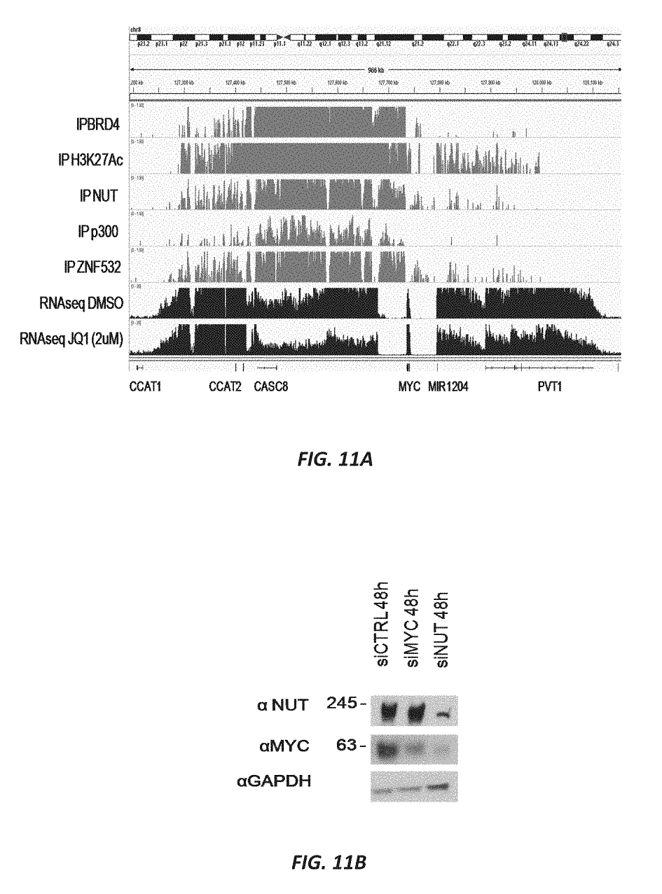

Differentiation in response to knockdown of ZNF532-NUT using both NUT- and ZNF532-specific siRNAs is demonstrated by FIG. 10A. Morphologic differentiation is evidenced by accumulation of abundant cytoplasm, enlargement of nuclei and cytoplasm, and formation of cohesive spheres. 234335 cells demonstrate similar sensitivity to BET inhibitor, JQ1, as TC-797 cells (FIG. 10C). It has been shown that an important function of ZNF532-NUT is to maintain expression of MYC. ChIP-seq has demonstrated that ZNF532-NUT occupies extremely large, 100 kb-2megabase, stretches of acetylated chromatin similar to the megadomains formed by BRD4-NUT (13). One such megadomain occupies a region proximal to MYC (FIG. 11A). RNA sequencing showed that this megadomain is associated with the BET-protein-dependent upregulation of the lncRNA, PVT1, that has been implicated in the stabilization of MYC protein (14). Treatment with JQ1 leads to decreased expression of the PVT1 RNA (FIG. 11A). Consistent with its role in MYC upregulation, knockdown of ZNF532-NUT leads to decreased MYC protein levels (FIG. 11B).

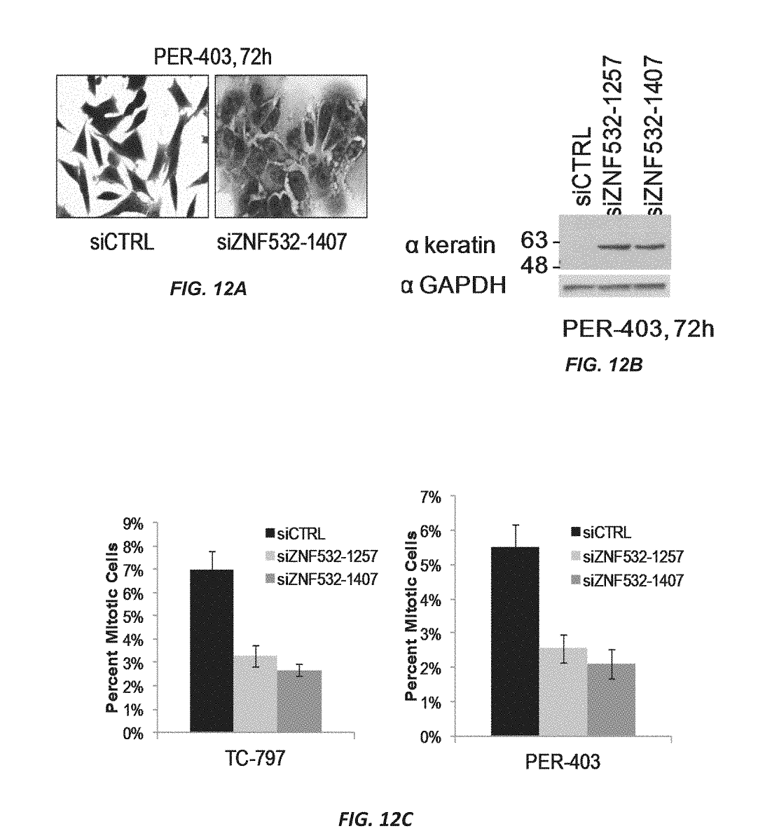

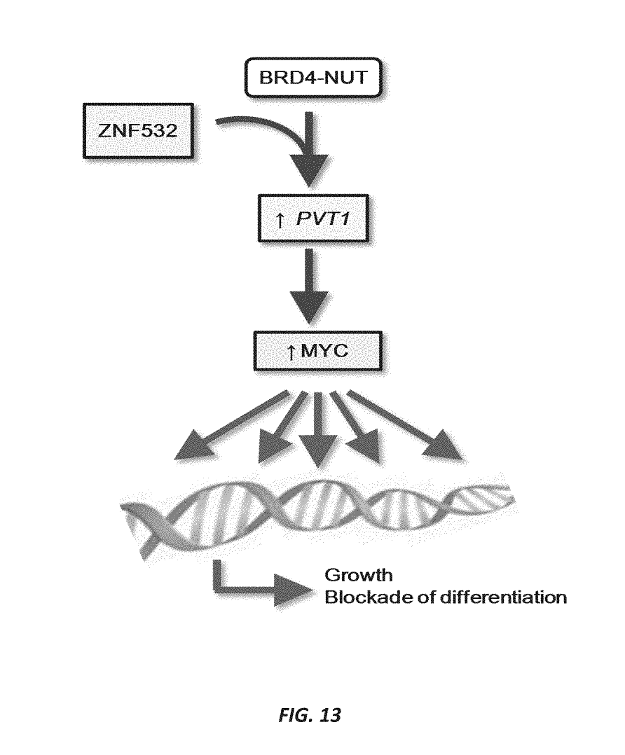

It is shown that 24335 cells are dependent upon BET proteins (FIG. 10C, 11A), which include BRD4. This is expected based on the uniform presence of BRD4 in all known NUT-fusion oncogene complexes (15). Mass spec of ZNF532-NUT expressed in 293T cells was performed and results were compared the complex components with those of BRD4-NUT (Table 3), and found that BRD4 is indeed present within the ZNF532-NUT complex. Moreover, multiple other common members of ZNF532- and BRD4-NUT complexes, including importantly wild tyepZNF532, other BET proteins and the histone acetyl transferases, p300/CBP were found. The overall findings demonstrate a similar function of ZNF532-NUT to form megadomains that drive transcription of key pro-growth, anti-differentiative genes, such as MYC. Thus it follows that wild type ZNF532 is likely required for BRD4-NUT function. The role of ZNF532 in BRD4-NUT function, required for the blockade of differentiation has been demonstrated in NMC cell line, PER-403 (FIG. 12A-B). Therefore, wild type ZNF532 is required for maintenance of proliferation of both of this cell line (FIG. 12C). Without wishing to be bound by theory, ZNF532 acts with BRD4-NUT to upregulate MYC expression by upholding expression of PVT1 (FIG. 13) and transcription factor ZNF532 plays a pivotal role in the localization of BRD4-NUT to the MYC megadomain in all NMCs. ZNF532 is required for the blockade of differentiation of non-NMC head and neck squamous cancers (HNSQC) cell lines, including the BICR6, BICR16, BHY, and Ca133 HNSQC cell lines, and have also shown that ZNF532 is required for the proliferation and invasiveness of these cell lines (FIG. 14).

### REFERENCES

1. French C A, Kutok J L, Faquin W C, Toretsky J A, Antonescu C R, Griffin C A, et al. Midline carcinoma of children and young adults with NUT rearrangement. J Clin Oncol. 2004; 22(20):4135-9.
2. French C A, Miyoshi I, Kubonishi I, Grier H E, Perez-Atayde A R, Fletcher J A. BRD4-NUT fusion oncogene: a novel mechanism in aggressive carcinoma. Cancer research. 2003; 63(2):304-7.
3. French C A, Ramirez C L, Kolmakova J, Hickman T T, Cameron M J, Thyne M E, et al. BRD-NUT oncoproteins: a family of closely related nuclear proteins that block epithelial differentiation and maintain the growth of carcinoma cells. Oncogene. 2008; 27(15):2237-42.
4. French C A, Rahman S, Walsh E M, Kuhnle S, Grayson A R, Lemieux M E, et al. NSD3-NUT Fusion Oncoprotein in NUT Midline Carcinoma: Implications for a Novel Oncogenic Mechanism. Cancer discovery. 2014; 4(8):928-41. Epub 2014/05/31.
5. Bauer D, Mitchell C, Strait K, Lathan C, Stelow E, Lueer S, et al. Clinicopathologic Features and Long-Term Outcomes of NUT Midline Carcinoma. Clinical cancer research: an official journal of the American Association for Cancer Research. 2012; 18(20):5773-9. Epub 2012/08/17.
6. Filippakopoulos P, Qi J, Picaud S, Shen Y, Smith W B, Fedorov 0, et al. Selective inhibition of BET bromodomains. Nature. 2010; 468(7327):1067-73. Epub 2010/09/28.
7. Alekseyenko A A, McElroy K A, Kang H, Zee B M, Kharchenko P V, Kuroda M I. BioTAP-XL: Cross-linking/Tandem Affinity Purification to Study DNA Targets, RNA, and Protein Components of Chromatin-Associated Complexes. Current protocols in molecular biology/edited by Frederick M Ausubel [et al]. 2015; 109:21 30 1-21 30 2. Epub 2015/01/07.
8. Rahman S, Sowa M E, Ottinger M, Smith J A, Shi Y, Harper J W, et al. The Brd4 extraterminal domain confers transcription activation independent of pTEFb by recruiting multiple proteins, including NSD3. Molecular and cellular biology. 2011; 31(13):2641-52. Epub 2011/05/11.
9. Dawson M A, Prinjha R K, Dittmann A, Giotopoulos G, Bantscheff M, Chan W I, et al. Inhibition of BET recruitment to chromatin as an effective treatment for MLL-fusion leukaemia. Nature. 2011; 478(7370):529-33. Epub 2011/10/04.
10. Reynoird N, Schwartz B E, Delvecchio M, Sadoul K, Meyers D, Mukherjee C, et al. Oncogenesis by sequestration of CBP/p300 in transcriptionally inactive hyperacetylated chromatin domains. The EMBO journal. 2010; 29(17):2943-52. Epub 2010/08/03.
11. Grayson A R W E, Cameron M J, Godec J, Ashworth T, Ambrose J M, Aserlind A B, Wang H, Evan G, Kluk M J, Bradner J E, Aster J C, French C A. MYC, a downstream target of BRD-NUT, is necessary and sufficient for the blockade of differentiation in NUT midline carcinoma. Oncogene. 2013
12. Toretsky J A, Jenson J, Sun C C, Eskenazi A E, Campbell A, Hunger S P, et al. Translocation (11; 15; 19): a highly specific chromosome rearrangement associated with poorly differentiated thymic carcinoma in young patients. Am J Clin Oncol. 2003; 26(3):300-6.
13. Alekseyenko A. A. WEM, Wang X., Grayson A. R., Hsi P. T., Kharchenko P. V., Kuroda M. I., French C. A. The oncogenic BRD4-NUT chromatin regulator drives aberrant transcription within large topological domains. Genes Dev. 2015; 29(14):1507-23.
14. Tseng Y Y, Moriarity B S, Gong W, Akiyama R, Tiwari A, Kawakami H, Ronning P, Reuland B, Guenther K, Beadnell T C, Essig J, Otto G M, O'Sullivan M G, Largaespada D A, Schwertfeger K L, Marahrens Y, Kawakami Y, Bagchi A. PVT1 dependence in cancer with MYC copy-number increase. Nature. 2014; 512(7512):82-6.
15. French C A, Rahman S, Walsh E M, Kuhnle S, Grayson A R, Lemieux M E, Grunfeld N, Rubin B P, Antonescu C R, Zhang S, Venkatramani R, Dal Cin P, Howley P M. NSD3-NUT Fusion Oncoprotein in NUT Midline Carcinoma: Implications for a Novel Oncogenic Mechanism. Cancer discovery. 2014; 4(8):928-41.
16. Stathis A, Zucca E, Bekradda M, Gomez-Roca C, Delord J P, de La Motte Rouge T, Uro-Coste E, de Braud F, Pelosi G, French C A*. Clinical Response of Carcinomas Harboring the BRD4-NUT Oncoprotein to the Targeted Bromodomain Inhibitor OTX015/MK-8628. Cancer Discovery. 2016 in press.

1. A method of treating cancer in a subject comprising administering a pharmaceutically-effective amount of a ZNF532 inhibitor to the subject.

2. The method of claim 1, wherein the cancer expresses ZNF532 protein or comprises a ZNF532-NUT fusion gene.

3. The method of claim 2, wherein the cancer is selected from the group consisting of NUT midline carcinoma (NMC), Ewing sarcoma, and head and neck squamous cell carcinoma.

4. The method of claim 1, wherein the ZNF532 inhibitor is selected from the group consisting of a small molecule, an antibody, an antibody fragment, RNAi, siRNA, a ZNF532 decoy molecule, a polypeptide that blocks the binding of ZNF532 with NUT and/or BRD4.

5. The method of claim 1, wherein the subject is a mammal.

6. The method of claim 5, wherein the mammal is a human.

7. A method of treating cancer in a subject comprising requesting a test to (i) measure a level of ZNF532 or (ii) determine whether a ZNF532-NUT fusion gene is present in a sample obtained from a subject, and administering to the subject a pharmaceutically-effective amount of a ZNF532 inhibitor and/or a BET inhibitor in response to the level of ZNF532 above a reference level.

8. (canceled)

9. The method of claim 7, wherein the cancer is selected from the group consisting of NUT midline carcinoma (NMC), Ewing sarcoma, and head and neck squamous cell carcinoma.

10. The method of claim 7, wherein the ZNF532 inhibitor is selected from the group consisting of a small molecule, an antibody, an antibody fragment, RNAi, siRNA, a ZNF532 decoy molecule, a polypeptide that blocks the binding of ZNF532 with NUT and/or BRD4.

11. The method of claim 7, wherein the BET inhibitor is selected from the group consisting of: JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), GSK-525762A, LY294002, 1-[2-(1/-/-benzimidazol-2-ylthio)ethyl]-1,3-dihydro-3-methyl-2H-benzinidazole-2-thione, 1-methylethyl ((2S,4R)-1-acetyl-2-methyl-6-{4-[(methylamino)methyl]phenyl}-1,2,3,4-tetrahydro-4-quinolinyl)carbamate, 2-[(4S)-6-(4-Chlorophenyl)-1-methyl-8-(methyloxy)-4H-[1,2,4]triazolo [4,3-a][1,4]benzodiazepin-4-yl]-N-ethylacetamide, 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one, 7-(3,5-dimethyl-4-isoxazolyl)-8-(methoxy)-1-[(1R)-phenylethyl]-2-(tetrahydro-2H-pyran-4-yl)-1H-imidazo[4,5-c]quinolone, 4-{(2S,4R)-1-acetyl-4-[(4-chlorophenyl)amino]-2-methyl-1,2,3,4-tetrahydro-6-quinolinyl}benzoic acid, and N-{1-methyl-7-[4-(1-piperidinylmethyl)phenyl][1,2,4]triazolo [4,3-a]quinolin-4-yl}urea, OTX015, CPI-0610, and Volasertib.

12. The method of claim 7, wherein the sample is a cancer sample.

13. The method of claim 7, wherein the subject is a mammal.

14. The method of claim 13, wherein the mammal is a human.

15.-16. (canceled)

17. A method of determining whether a BET inhibitor therapy can be effective in a subject having cancer, the method comprising:
(i) measuring, in a sample obtained from the subject, a level of ZNF532 or determining whether a ZNF-532-NUT fusion gene is present in the sample; and
(ii) ii determining that the therapy can be effective if the level of ZNF532 is above a reference level or the ZFN-NUT fusion gene is present in the sample.

18. The method of claim 17, further comprising administering the BET inhibitor therapy to the subject when the level of ZNF532 is above the reference level.

19. (canceled)

20. The method of claim 17, further comprising administering the BET inhibitor therapy to the subject when the ZNF532-NUT fusion gene is present in the sample.

21. The method of claim 17, wherein the sample is a cancer sample.
